(19) United States
(12) Patent Application Publication
(10) Pub. No.: US 2022/0243267 A1
(43) Pub. Date: Aug. 4, 2022

(54) COMPOSITIONS AND METHODS RELATED TO QUANTITATIVE REDUCED REPRESENTATION SEQUENCING

(71) Applicants: North Carolina State University, Raleigh, NC (US); University of Tennessee Research Foundation, Knoxville, TN (US)

(72) Inventors: George C. YENCHO, Raleigh, NC (US); Bode A. OLUKOLU, Knoxville, TN (US)

(73) Assignees: North Carolina State University, Raleigh, NC (US); University of Tennessee Research Foundation, Knoxville, TN (US)

(21) Appl. No.: 17/614,948

(22) PCT Filed: May 30, 2020
(86) PCT No.: PCT/US2020/035470

§ 371 Date: Nov. 29, 2021

Related U.S. Application Data
(60) Provisional application No. 62/855,108, filed on May 31, 2019.

(51) Int. Cl. C12Q 1/6874; C12Q 1/6806

(52) U.S. Cl. CPC C12Q 1/6874; C12Q 1/6806

(57) ABSTRACT

The present disclosure provides compositions and methods pertaining to a next-generation sequencing (NGS) library preparation protocol and method for the optimization of sequencing quality and yield. In particular, the present disclosure provides a novel sequencing platform referred to as OmeSeq, which enables high-fidelity, dosage-sensitive genotyping and strain-level metagenomic profiling of various DNA and RNA templates across animal, plant, microbial, and viral genomes.

### CROSS REFERENCE TO RELATED APPLICATION

This application claims priority to and the benefit of U.S. Provisional Patent Application No. 62/855,108 filed May 31, 2019, which is incorporated herein by reference in its entirety and for all purposes.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 2,000 Byte ASCII (Text) file named “38417-601_ST25,” created on May 30, 2020.

### FIELD

The present disclosure provides compositions and methods pertaining to a next-generation sequencing (NGS) library preparation protocol and method for the optimization of sequencing quality and yield. In particular, the present disclosure provides a novel sequencing platform referred to as OmeSeq, which enables quantitative, high-fidelity, dosage-sensitive genotyping and strain-level metagenomic profiling of various DNA and RNA templates across animal, plant, microbial, and viral genomes.

### BACKGROUND

The massively parallel sequencing technology known as next-generation sequencing (NGS) has revolutionized the biological sciences. With its ultra-high throughput, scalability, and speed, NGS enables researchers to perform a wide variety of applications and study biological systems at a level never before possible. Current complex genomic research questions generally demand a depth of information beyond the capacity of traditional DNA sequencing technologies. Next-generation sequencing has filled that gap and become an everyday research tool to address these questions. Additionally, innovative sample preparation and data analysis options enable a broad range of applications. For example, NGS allows researchers to rapidly sequence whole genomes, focus in to deeply sequence target regions, utilize RNA sequencing (RNA-Seq) to discover novel RNA variants and splice sites and quantify mRNAs for gene expression analysis, analyze epigenetic factors such as genome-wide DNA methylation and DNA-protein interactions, sequence disease samples to study rare somatic variants, tumor subclones, and study microbial diversity in humans or in the environment.

Genetic polymorphisms, particularly single nucleotide polymorphisms (SNPs), have been widely used to advance quantitative, functional and evolutionary genomics. Ideally, all genetic variants among individuals would be discovered when NGS technologies and platforms are used for whole genome sequencing or resequencing. However, in order to improve the cost-effectiveness of the process, the research community has mainly focused on developing genome-wide sampling sequencing (GWSS) methods, a collection of reduced genome complexity sequencing, reduced genome representation sequencing and selective genome target sequencing. To address the current limitations associated with SNP arrays/chips and the high/low coverage of whole genome sequencing/resequencing platforms, the genome research community has been developing alternative strategies to discover and genotype genetic variants in a cost effective manner. Basically, these alternative methods/techniques are NGS-based, but different laboratory procedures can result in different data outcomes in terms of reduced genome complexities, reduced genome representations, or selected genome targets. Currently available GWSS methods have mainly evolved from reduced representation (library) sequencing (RRS or RRLS), complexity reduction of polymorphism sequencing (CRoPS™) restriction site associated DNA sequencing (RAD-seq), and genotyping by sequencing (GBS) methods.

### SUMMARY

Embodiments of the present disclosure provide forward and reverse single-stranded DNA (ssDNA) adapter molecules for use in quantitative reduced representative sequencing (qRRS). In accordance with these embodiments, the ssDNA adapters include: (i) a probe binding region at the 5′ end of the adapters; (ii) a buffer region distal to the probe binding region; (iii) a barcode region distal to the buffer region; and (iv) a restriction enzyme overhang motif at the 3′ end of the adapters.

In some embodiments, the restriction enzyme overhang motif comprises a nucleic acid sequence complementary to an overhang sequence produced upon cleavage by a restriction enzyme. In some embodiments, the adapters are bound to a fragment of genomic DNA via complementation between the restriction enzyme motif of the ssDNA adapters and the genomic DNA produced upon cleavage by the restriction enzyme. In some embodiments, the restriction enzyme produced a 5′ overhang. In some embodiments, the restriction enzyme is NsiI or NlaIII.

In some embodiments, the buffer region comprises a nucleic acid sequence from 4 to 8 base pairs in length. In some embodiments, the buffer region comprises a nucleic acid sequence that is 6 base pairs in length.

In some embodiments, the barcode region comprises a nucleic acid sequence from 5 to 12 base pairs in length. In some embodiments, the barcode region comprises a nucleic acid sequence from 7 to 10 base pairs in length.

In some embodiments, the buffer region is directly adjacent to the barcode region. In some embodiments, the barcode region is directly adjacent to the restriction enzyme motif. In some embodiments, the probe binding region facilitates binding to a substrate or probe. In some embodiments, the probe binding region facilitates binding to a separate nucleic acid molecule that is complementary to at least a portion of the nucleic acid sequence of the probe binding region. In some embodiments, the total length of the adaptor is from 25 to 100 base pairs.

Embodiments of the present disclosure also include a kit comprising any of the ssDNA adapters described above. In accordance with these embodiments, the kit can be used to perform a sequencing reaction. In some embodiments, the kit further comprises at least one of a buffer, dNTPs, a polymerase, a restriction enzyme, and/or cos-probes or pooled cos-probes.

Embodiments of the present disclosure also include a double-stranded genomic DNA fragment comprising the ssDNA adapter molecules described above appended to each end of the genomic DNA fragment. In accordance with these embodiments, the present disclosure also includes a composition comprising a plurality of genomic fragments comprising the ssDNA adapter molecules described above.

Embodiments of the present disclosure also include a solution-based array composition. In accordance with these embodiments, the array composition includes a plurality of DNA complementary overhanging sequence probes (cos-probes) capable of integration into targeted regions of a genomic template, and any of the ssDNA adapters described above. In some embodiments, the cos-probes include at least one hairpin structure and an overhang complementary to the 5′ overhang of the restriction enzyme motif.

Embodiments of the present disclosure also include a quantitative reduced representation sequencing (qRRS) method. In accordance with these embodiments, the method includes: (i) appending the ssDNA adapter molecules of any of claims 1 to 12 to a plurality of nucleic acid fragments digested to form a nucleic acid library; (ii) amplifying the plurality of nucleic acid fragments in the library using PCR and/or isothermal amplification; (iii) hybridizing the library to a nucleic acid sequencing platform; and (iv) sequencing the genomic fragments.

In some embodiments, the nucleic acids fragments have been digested with a restriction enzyme. In some embodiments, the nucleic acid fragments are RNA or DNA molecules. In some embodiments, appending the ssDNA adapter molecules comprises the use of cos-probes.

In some embodiments, the method results in at least 25% more sequencing reads. In some embodiments, the method results in at least 50% more sequencing reads. In some embodiments, the method comprises multiplexing. In some embodiments, the method removes chimeric fragments caused by reconstitution of restriction enzyme sites. In some embodiments, the method does not comprise PCR or ligation reactions. In some embodiments, the method minimizes barcode swapping. In some embodiments, the method enhances cluster generation. In some embodiments, the method comprises quantification of allele dosage in diploid and polyploid organisms. In some embodiments, the method comprises an error rate of less than 0.0002 across an entire length of a read, including proximal and distal ends that typically have high error rates.

In some embodiments of the method, the genomic DNA is obtained from one or more of bacteria, viruses, protozoa, plants, fungi, yeast, mammals, and any combination thereof. In some embodiments, the genomic DNA is obtained from a metagenome. In some embodiments, the genomic DNA is obtained from a microbiome. In some embodiments, the genomic DNA is obtained from an organism having a polyploid genotype.

### BRIEF DESCRIPTION OF THE DRAWINGS

FIGS. 1A-1D: (A) Representative schematic diagram of exemplary designs of the ssDNA adapter molecules of the present disclosure for both single-end and paired-sequencing. The dual-barcoding allows for a multiplexed assay of 9,216 pooled samples during paired-end sequencing. (B) A representative schematic diagram of the OmeSeq (e.g., qRRS) next-generation sequencing library preparation workflow. (C) A representative schematic diagram of the OmeSeq-Array next-generation sequencing library preparation workflow. (D) A representative schematic diagram of the OmeSeq-noSeq (no requirement for sequencing) library preparation workflow.

FIG. 2: Representative consistent median quality scores at maximum on platform (Q37), including buffer and barcode sequences. Boxplot shows blue dash as median; absence of boxes indicative of minimal variation around median; absence whiskers indicating minimal/no outliers; and grey diamonds as the mean. Results demonstrate increased yields due to flow cell cluster enhancing methods (e.g., Illumina maximum number of reads at 1.6 billion reads vs. OmeSeq's 55% more reads at 2.476 billion reads).

FIG. 3: Representative metrics showing the performance of qRRS compositions and methods of the present disclosure (OmeSeq) using highly degraded DNA samples that have failed clustering and sequencing with several other library preparation methods. The combination of a DNA repair step with OmeSeq delivers accurate base calls, 20% more yield, even representation of pooled samples independent of DNA quality, and the ability to map almost 99% of the reads to a draft reference genome.

FIGS. 4A-4C: QC plot of low quality NGS data based on suboptimal protocol parameters. (A) Diagram of adapter-contaminated reads displaying buffer region, barcode, and restriction sites, as well as the corresponding reverse-complement adapter regions. (B) Boxplots show the lower Q scores 5′ and 3′ ends. (C) Read length density after adapter removal using ngsComposer pipeline. Adapter only (red), adapter through barcode (blue), and adapter through restriction site (yellow) each show different performance in adapter detection.

FIG. 5: Comparison of sample assignment (demultiplexing) of multiplexed/pooled samples during next-generation sequencing. Several tools are influenced by the order (top and bottom figure) of barcodes when searching for potential matches (ea-utils and sabre). In some instances, tools will preferentially reassign reads once mismatch is increasing across columns from left to right. Values within the heat map indicate the degree of deviation computed as proportion of reads at mismatch 0, which is specified within each tool, relative to after allowing mismatch. The midpoint, yellow, indicates zero deviation.

FIG. 6: Quality scores from 4 Illumina platforms measured within barcode regions of reads. The empirical rate of base-calling errors (squares) were sequentially calculated using increasing Hamming distance in the ngsComposer demultiplexing tool, anemone. The Q scores for these bases reported by Illumina software reveal underestimation of base-calling error. Open shapes indicate the mean Q scores, while solid shapes indicate Q scores for individual base positions along the barcode region.

FIG. 7: Sample results using Q score threshold-filtering (reads with 95% of reads haves base calls at Q-30; a very strict filtering parameter and likely inflated false negative rate) and motif-based filtering on the Hiseq 2500 Rapid Run dataset (122,317,249 reads; low-quality sequence dataset). Inner and outer rings represent proportion of reads passing the first and second filtering tools, respectively. Exterior labels are the compression rates of reads that pass and fail filtering in the 2-step filtering approach. Reads passing both tools have the highest compression values indicating a reduction in sequencing error. Both filtering methods detect erroneous reads that are non-overlapping, hence, underscoring the need to both strategies.

FIGS. 8A-8C: QC plots of low-quality (A) and high-quality (B) sequence dataset of R2 reads, which contains more error. Motif-based error detection and removal algorithm implemented in the software Rotifer (a component of the ngsComposer pipeline). Reads that fail filtering by Rotifer (e.g., using the restriction site motif at beginning of each read) revealed propagation of base calling error along entire length of reads. (C) QC plot of raw reads from optimal OmeSeq/qRRS-derived dataset.

FIGS. 9A-9C: (A) Shotgun species-level benchmarking (red: recall rate) reveals that Qmatey outperforms existing tools. (B) Phylogeny/taxonomic composition present in leaf microbiome of at least 5% of 767 sweetpotato accessions. Attempt to confirm taxa (species or strain) in literature (green and yellow). (C) Qmatey's analytical workflow.

FIGS. 10A-10C: (A) strain-level profile reveals microbe-microbe interactions based on leaf microbiome (generated using OmeSeq/qRRS) of 767 sweetpotato accessions. Positive (blue) and negative (red) values indicate potential synergistic and antagonistic interactions, respectively. (B) microbe-microbe interactions, subset of FIG. 10A, reveals multipartite interactions that might modulate resistance to Fusarium. These involve whitefly (Bemisia tabaci), whitefly endosymbiont, sweetpotato viruses transmitted by whiteflies, and insect interaction with entomopathogenic Fusarium. (C) Manhattan plots show examples of significant loci controlling specific host-associated viruses and fungus, and biocontrol within the microbiome (all of which were validated by literature). High-density SNPs were generated using OmeSeq/qRRS.

FIGS. 11A-11B: K-means clustering of 767 sweetpotato accessions based on quantitative profiles of metagenome associated with each accession (A) and based on the high-density SNP data (B). Clustering pattern reveals that individual host-microbiome composition is driven by a genetic architecture independent of shared ancestry within sub-populations. Clustering of accessions based on microbiome composition suggest recruitment of some member microbes are evolutionary conserved. Nevertheless, variation exist within some accessions that diverge from this consensus.

FIGS. 12A-12C: The taxonomic profiles of the sweetpotato diversity panel, including down to strain level (A), species (B), and genus (C) level profiling. Each panel includes pair-wise spearman correlations of each taxonomic match within the profile, which reflect the superior capability of the strain-level profiling to detect signals underlying functional multipartite interactions among members of the community. Most of the correlations are positive, indicating the communities have co-evolved and are conserved to a large extent across the sweetpotato germplasm. The relative limited number of species observed (B) agrees with other studies that generally reveal a low diversity in leaf microbiome across plants.

FIG. 13: Correlation plot depicting Pearson Correlation coefficients between sugar profile traits.

FIG. 14: Manhattan plots (left) showing all gene dosage models for SNP associations with glucose profile in baked sweetpotato storage roots. Q-Q plots (right) for reveals the power of detection and extent of false positive rate for all gene dosage models.

FIG. 15: Manhattan plots (left) showing all gene dosage models for SNP associations with glucose profile in raw sweetpotato storage roots. Q-Q plots (right) for reveals the power of detection and extent of false positive rate for all gene dosage models.

FIG. 16: Manhattan plots (left) showing all gene dosage models for SNP associations with fructose profile in baked sweetpotato storage roots. Q-Q plots (right) for reveals the power of detection and extent of false positive rate for all gene dosage models.

FIG. 17: Manhattan plots (left) showing all gene dosage models for SNP associations with fructose profile in raw sweetpotato storage roots. Q-Q plots (right) for reveals the power of detection and extent of false positive rate for all gene dosage models.

FIG. 18: Manhattan plots (left) showing all gene dosage models for SNP associations with maltose profile in baked sweetpotato storage roots. Q-Q plots (right) for reveals the power of detection and extent of false positive rate for all gene dosage models.

FIG. 19: Manhattan plots (left) showing all gene dosage models for SNP associations with maltose profile in raw sweetpotato storage roots. Q-Q plots (right) for reveals the power of detection and extent of false positive rate for all gene dosage models.

### DETAILED DESCRIPTION

Embodiments of the present disclosure provide compositions and methods pertaining to a quantitative next-generation sequencing (NGS) library preparation protocol and method for the optimization of sequencing quality and yield. In particular, embodiments of the present disclosure provide a novel sequencing platform referred to as OmeSeq, which enables high-fidelity, dosage-sensitive genotyping and strain-level metagenomic profiling of various DNA and RNA templates across animal, plant, microbial, and viral genomes.

Massively parallel DNA sequencing is now a pervasive tool; however, many next-generation sequencing methods and platforms have not been able to overcome significant challenges, which limit their utility. To address these challenges, and others, embodiments of the present disclosure include a significant advancement in short-read next-generation library preparation and sequencing. In accordance with these embodiments, the present disclosure provides novel compositions, methods, and systems for high-fidelity, dosage-sensitive genotyping and quantitative strain-level metagenomic profiling (OmeSeq). The scalable, ligation-free and PCR-free assay platform (e.g., a 9,216-multiplexed assay format, a 147,456-multiplexed assay format, and the like) reduces off-target hybridization by using single-stranded adapters for isothermal strand displacement of dsDNA with 4 bp overhangs as priming sites. Novel features of the OmeSeq platform described further herein are amenable to many applications, including but not limited to, whole genome, transcriptome, and methylome sequencing. Some of these features include a paradigm shift in adapter design that prevents chimeric reads and barcode swapping, a flow cell cluster enhancer that generates about 50% more yields, and consistent high-quality scores across all base positions. Additionally, the workflow is optimized for ease-of-use and requires two days of preparation.

The features of OmeSeq as described further herein are applicable to many sequencing platforms and methodologies, including but not limited to, (i) reduced representation sequencing (RRS); (ii) shotgun whole genome and metagenome sequencing; (iii) full-length and partial cDNA sequencing of transcriptomes and meta-transcriptomes; and (iv) other specialized applications such as methylome sequencing. The quantitative RRS (qRRS) methods and compositions described herein can be used for high-throughput genome-wide marker genotyping, metagenome profiling, high-throughput in solution array-based targeted-assays (OmeSeq-Array), and low-through targeted-assays (OmeSeq-noSeq). Among other advantages, OmeSeq-Array is also designed to reduce ascertainment bias using in-situ sequence filtering.

The use of OmeSeq for qRRS provides a scalable and flexible assay platform that uses next-generation and massively paralleled sequencing to quantitatively capture allele dosage during variant/SNP genotyping in diploid and polyploid organisms, without a need for data imputation due to minimal data missingness and low allelic dropout. This is particularly crucial for polyploids that lack an effective genotyping platform. While attempts have been made to use SNP chips, the scientific community has had little success accurately measuring allelic ratios. Consequently, polyploid genotypes are often diploidized.

The OmeSeq qRRS compositions and methods of the present disclosure also provide quantitative profiling and strain-level taxonomic identification of organisms (e.g., viruses, bacteria, protozoa, fungi and other eukaryotes) within a metagenomic/microbiome community. While two major methods already exist for metagenomic/microbiome profiling (i.e. amplicon and shotgun sequencing), their applications are limited. Amplicon sequencing platforms are usually based on a single gene (e.g., the rRNA gene) that lacks resolution for species- or strain-level identification. Because of this, for example, organisms are often identified with presumed operational taxonomic units that clusters organisms within the genus level. Consequently, quantification is beyond the scope of amplicon sequencing assays. While metagenomic shotgun sequencing platforms have the potential to deliver strain-level identification and quantification, they are cost-prohibitive and computationally intensive.

As described further herein, embodiments of the present disclosure can be used to extend qRRS methodology, resulting in a targeted in-solution assay (“in-solution” OmeSeq, in which hybridization reactions occur in an aqueous phase as oppose to a solid phase (e.g., silicon chip) used in a conventional SNP chip/array). This methodology represents an important improvement over current methods, since a blind sequencing of various regions of the genome does not always lead to diagnostic or informative sequences (e.g., a significant portion of the sequences are the same across multiple individuals, hence, reads are wasted on non-informative sequences). In contrast, the OmeSeq Array involves the targeted sequencing of informative sequences with the ability to reduce cost and focus on gene-based diagnostics within a group of species or strains. OmeSeq Array is also a powerful methodology for targeting endophytic microbiomes since the community continues struggle with the challenges of excluding the host DNA that comprises over 95% of the metagenome.

As described further herein, embodiments of the present disclosure include forward and reverse single-stranded DNA adapter molecules comprising a 4-8 base pair (e.g., 6 base pair) buffer sequence or region upstream of variable length barcode regions, which ensures that the barcodes used for demultiplexing pooled samples are shifted into regions of high base call rate since the proximal and distal ends of reads tend to have higher base calling error rates. This significantly reduces base calling error, a major reason for barcode swapping or sample misassignment. While each base position in the buffer sequence can be degenerate, this is not optimal. The buffer sequences used in the ssDNA adapters described herein are designed to optimize and maintain the required nucleotide diversity for short-read sequencing, and ensure that assay restriction sites (e.g., NsiI: ATGCAT; and NlaIII: CATG) are not created since the presence of these motifs will lead to loss of barcoded samples, partial failure of assay, and an unbalanced library, which will subsequently lead to high error rates in base calls. Furthermore, use of degenerate sequences that lack design will also produce repeats that make sequencing platforms (e.g., Illumina platforms) prone to indel error and phasing error.

The variable length barcodes were also designed to include various features that prevent the presence of chimeric reads in an NGS library. Although variable length barcode regions that maintain nucleotide diversity have been used, the barcode regions included in the ssDNA adapters of the present disclosure include variable length barcode sequences that destroy restriction sites upon integration at the left adapter-genomic fragment junction. Previous methods often form chimeric fragments by re-constitution of restriction site (e.g., ligation-based methods) and by partial extension-derived fragments that act as primers on off-target genomic regions (e.g., PCR-based methods). By performing a secondary digest, chimeric fragments are eliminated but constructs derived from the adapters of the present disclosure lack the restriction site and, consequently, remain intact. The concept of destroying restriction sites in fragments of interest can be applied to ligation-based methods as well.

To prevent chimeric fragments derived from PCR-based methods, the compositions and methods of the present disclosure implement a novel feature termed “double-stranded-based template protection,” which prevents off-target hybridization. This double-stranded-based template protection feature prevents off-target hybridization that is typical of PCR-based assays. The assay platforms of the present disclosure maintain the double-stranded secondary structure of DNA template and avoids DNA denaturing during incorporation of adapters. The 3′-overhang produced after digesting the genome is the only portion of the fragment accessible to the single-stranded adapter for strand displacement and isothermal 5′-to-3′ strand synthesis.

After isothermal 5′-to-3′ strand synthesis, the displaced strand is retained within the library preparation and serves as a cluster generation enhancer, for example, on an Illumina flow cell platform, which leads to the generation of as much as 55% more reads than the maximum reported. Since the displaced fragment is a perfect complement of the sequence-able genomic template in the adapter-template hybrid, the displaced fragment also serves as a primer during the incorporation of universal sequences complementary to the two probes on the Illumina flow cell. While the left (P5) and right (P7) probe sequence complements are integrated on both sides of the sequence-able adapter-template hybrid, the displaced fragments are extended only in the 5′-to-3′ direction so that they only incorporate 1 of the 2 probe sequence complements. Although these fragments derived from the displaced fragments also bind the flow cell probes, they are not compatible with bridge-amplification, which is required for cluster formation. Nevertheless, their binding leads to higher definition and resolution of clusters. Although the concentration of sequence-able fragments (e.g., contains both probe sequence complements) are optimized so that they are spaced out and only one molecule forms a cluster, this is not always the case. Some fragments bind probes and undergo bridge-amplification within proximity, which results in mixed and poor cluster signal and consequently cluster failure. If two fragments are within proximity, the cluster enhancing strategy provided herein increases the odds that only one of them will form bridge-amplification and consequently a well-defined cluster signal. The result is an increased density of clusters with clean signals, an increase in cluster passing filter, and a consequent increase in read yield by as much as 55%.

As an extension of the OmeSeq/qRRS compositions and methods of the present disclosure, embodiments were also developed to provide an inexpensive targeted sequencing strategy, referred to as an in-solution OmeSeq-Array, that is rapidly developed for in-solution assays. It is comparable to SNP chips but does not require designing an array of probes on a physical chip. This methodology represents a paradigm shift in probe design to make development and deployment less challenging for various organisms. This provides quantitative genotyping and diagnostics at all taxonomic levels (e.g., from viruses to higher eukaryotic organisms).

Embodiments of the present disclosure also include the use of complementary overhanging sequences, also referred to as cos-probes, that adapt the similar strategy of double-stranded-based probe protection. To build a library of target regions of a genome, the plus-strand of restriction enzyme digested dsDNA is selectively degraded to single nucleotides and the minus ssDNA is used as a template for isothermal amplification. The use of cos-probes ensures that the probe will only anneal to the target at the proximal and distal ends of the target ssDNA at a stringent temperature of 65° C. This strategy prevents the temperature-dependent off-target hybridization and biases, which are weaknesses of existing methods. The in-solution OmeSeq-Array can be either coupled to a next-generation sequencing platform or resolved on platforms that do not require sequencing, OmeSeq-noSeq, which is more amenable to low-throughput assays (e.g., about a 50 SNP/sequence panel) that require a fast turn-around time.

Another component of the high-throughput OmeSeq-Array and the low-throughput OmeSeq-noSeq is the ability to design high-fidelity cos-probes rapidly and a novel multiplexed oligo-synthesis strategy that leads to significant reduction in cost. A haplotype-based SNP filtering protocol implemented during the discovery phase ensures that SNP/sequences are single copy within the genome, hence, a high SNP conversion rate and minimal data missingness. Due to the ds-DNA probe protection, the high-specificity of the cos-probes can be maintained while targeting only about 12 base pairs of genomic regions. Compared to current sequence capture methods that require about 100 base pair probes, the short probes lead to further reduction in the cost of an array/SNP panel. Because the assay does not depend on an annealing temperature, there are less constraints associated with designing primers and probes that are temperature dependent.

Section headings as used in this section and the entire disclosure herein are merely for organizational purposes and are not intended to be limiting.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences for the production of a polypeptide, precursor, or RNA (e.g., rRNA, tRNA, sRNA, microRNA, lincRNA). The polypeptide can be encoded by a full-length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc.). Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed).

As used herein, a “double-stranded nucleic acid” may be a portion of a nucleic acid, a region of a longer nucleic acid, or an entire nucleic acid. A “double-stranded nucleic acid” may be, e.g., without limitation, a double-stranded DNA, a double-stranded RNA, a double-stranded DNA/RNA hybrid, etc. A single-stranded nucleic acid having secondary structure (e.g., base-paired secondary structure) and/or higher order structure comprises a “double-stranded nucleic acid”. For example, triplex structures are considered to be “double-stranded”. In some embodiments, any base-paired nucleic acid is a “double-stranded nucleic acid”

The term “single-stranded” oligonucleotides generally refers to those oligonucleotides that contain a single covalently linked series of nucleotide residues.

The terms “oligomers” or “oligonucleotides” include RNA or DNA sequences of more than one nucleotide in either single chain or duplex form and specifically includes short sequences such as dimers and trimers, in either single chain or duplex form, which can be intermediates in the production of the specifically binding oligonucleotides. “Modified” forms used in candidate pools contain at least one non-native residue. “Oligonucleotide” or “oligomer” is generic to polydeoxyribonucleotides (containing 2′-deoxy-D-ribose or modified forms thereof), such as DNA, to polyribonucleotides (containing D-ribose or modified forms thereof), such as RNA, and to any other type of polynucleotide which is an N-glycoside or C-glycoside of a purine or pyrimidine base, or modified purine or pyrimidine base or abasic nucleotides. Oligonucleotide” or “oligomer” can also be used to describe artificially synthesized polymers that are similar to RNA and DNA, including, but not limited to, oligos of peptide nucleic acids (PNA).

As used herein, a “non-native” nucleic acid sequence refers to a nucleic acid sequence not normally present in a bacterium, e.g., an extra copy of an endogenous sequence, or a heterologous sequence such as a sequence from a different species, strain, or substrain of bacteria, or a sequence that is modified and/or mutated as compared to the unmodified sequence from bacteria of the same subtype. In some embodiments, the non-native nucleic acid sequence is a synthetic, non-naturally occurring sequence. The non-native nucleic acid sequence may be a regulatory region, a promoter, a gene, and/or one or more genes in a gene cassette. In some embodiments, “non-native” refers to two or more nucleic acid sequences that are not found in the same relationship to each other in nature. The non-native nucleic acid sequence may be present on a plasmid or chromosome. In addition, multiple copies of any regulatory region, promoter, gene, and/or gene cassette may be present in the bacterium, wherein one or more copies of the regulatory region, promoter, gene, and/or gene cassette may be mutated or otherwise altered as described herein. In some embodiments, the genetically engineered bacteria are engineered to comprise multiple copies of the same regulatory region, promoter, gene, and/or gene cassette in order to enhance copy number or to comprise multiple different components of a gene cassette performing multiple different functions.

As used herein, “promoter” refers to a nucleotide sequence that is capable of controlling the expression of a coding sequence or gene. Promoters are generally located 5′ of the sequence that they regulate. Promoters may be derived in their entirety from a native gene, or be composed of different elements derived from promoters found in nature, and/or comprise synthetic nucleotide segments. Those skilled in the art will readily ascertain that different promoters may regulate expression of a coding sequence or gene in response to a particular stimulus, e.g., in a cell- or tissue-specific manner, in response to different environmental or physiological conditions, or in response to specific compounds. Prokaryotic promoters are typically classified into two classes: inducible and constitutive.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (the oligonucleotide or polynucleotide may be single-stranded), but may contain both the sense and anti-sense strands (the oligonucleotide or polynucleotide may be double-stranded).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample. The term “substantially purified” as used herein refers to a molecule such as a polypeptide, carbohydrate, nucleic acid etc. which is substantially free of other proteins, lipids, carbohydrates or other materials with which it is naturally associated. One skilled in the art can purify viral or bacterial polypeptides using standard techniques for protein purification. The substantially pure polypeptide will often yield a single major band on a non-reducing polyacrylamide gel. In the case of partially glycosylated polypeptides or those that have several start codons, there may be several bands on a non-reducing polyacrylamide gel, but these will form a distinctive pattern for that polypeptide. The purity of the viral or bacterial polypeptide can also be determined by amino-terminal amino acid sequence analysis. Other types of antigens such as polysaccharides, small molecule, mimics etc. are included within the present disclosure.

“Peptide” and “polypeptide” as used herein, and unless otherwise specified, refer to polymer compounds of two or more amino acids joined through the main chain by peptide amide bonds (—C(O)NH—). The term “peptide” typically refers to short amino acid polymers (e.g., chains having fewer than 25 amino acids), whereas the term “polypeptide” typically refers to longer amino acid polymers (e.g., chains having more than 25 amino acids).

As used herein, the term “fragment” refers to a peptide or polypeptide that results from dissection or “fragmentation” of a larger whole entity (e.g., protein, polypeptide, enzyme, etc.), or a peptide or polypeptide prepared to have the same sequence as such. Therefore, a fragment is a subsequence of the whole entity (e.g., protein, polypeptide, enzyme, etc.) from which it is made and/or designed. A peptide or polypeptide that is not a subsequence of a preexisting whole protein is not a fragment (e.g., not a fragment of a preexisting protein).

As used herein, the term “sequence identity” refers to the degree two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) have similar polymer sequences. For example, similar amino acids are those that share the same biophysical characteristics and can be grouped into the families, e.g., acidic (e.g., aspartate, glutamate), basic (e.g., lysine, arginine, histidine), non-polar (e.g., alanine, valine, leucine, isoleucine, proline, phenylalanine, methionine, tryptophan) and uncharged polar (e.g., glycine, asparagine, glutamine, cysteine, serine, threonine, tyrosine). The “percent sequence identity” (or “percent sequence similarity”) is calculated by: (1) comparing two optimally aligned sequences over a window of comparison (e.g., the length of the longer sequence, the length of the shorter sequence, a specified window), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

In some embodiments the substitutions can be conservative amino acid substitutions. Examples of conservative amino acid substitutions, unlikely to affect biological activity, include the following: alanine for serine, valine for isoleucine, aspartate for glutamate, threonine for serine, alanine for glycine, alanine for threonine, serine for asparagine, alanine for valine, serine for glycine, tyrosine for phenylalanine, alanine for proline, lysine for arginine, aspartate for asparagine, leucine for isoleucine, leucine for valine, alanine for glutamate, aspartate for glycine, and these changes in the reverse. See e.g. Neurath et al., The Proteins, Academic Press, New York (1979), the relevant portions of which are incorporated herein by reference. Further, an exchange of one amino acid within a group for another amino acid within the same group is a conservative substitution, where the groups are the following: (1) alanine, valine, leucine, isoleucine, methionine, norleucine, and phenylalanine: (2) histidine, arginine, lysine, glutamine, and asparagine; (3) aspartate and glutamate; (4) serine, threonine, alanine, tyrosine, phenylalanine, tryptophan, and cysteine; and (5) glycine, proline, and alanine.

The term “homology” and “homologous” refers to a degree of identity. There may be partial homology or complete homology. A partially homologous sequence is one that is less than 100% identical to another sequence.

As used herein, the terms “complementary” or “complementarity” are used in reference to polynucleotides (e.g., a sequence of nucleotides such as an oligonucleotide or a target nucleic acid) related by the base-pairing rules. For example, for the sequence “5′-A-G-T-3′” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids. Either term may also be used in reference to individual nucleotides, especially within the context of polynucleotides. For example, a particular nucleotide within an oligonucleotide may be noted for its complementarity, or lack thereof, to a nucleotide within another nucleic acid strand, in contrast or comparison to the complementarity between the rest of the oligonucleotide and the nucleic acid strand.

In some contexts, the term “complementarity” and related terms (e.g., “complementary”, “complement”) refers to the nucleotides of a nucleic acid sequence that can bind to another nucleic acid sequence through hydrogen bonds, e.g., nucleotides that are capable of base pairing, e.g., by Watson-Crick base pairing or other base pairing. Nucleotides that can form base pairs, e.g., that are complementary to one another, are the pairs: cytosine and guanine, thymine and adenine, adenine and uracil, and guanine and uracil. The percentage complementarity need not be calculated over the entire length of a nucleic acid sequence. The percentage of complementarity may be limited to a specific region of which the nucleic acid sequences that are base-paired, e.g., starting from a first base-paired nucleotide and ending at a last base-paired nucleotide. The complement of a nucleic acid sequence as used herein refers to an oligonucleotide which, when aligned with the nucleic acid sequence such that the 5′ end of one sequence is paired with the 3′ end of the other, is in “antiparallel association.” Certain bases not commonly found in natural nucleic acids may be included in the nucleic acids of the present disclosure and include, for example, inosine and 7-deazaguanine. Complementarity need not be perfect; stable duplexes may contain mismatched base pairs or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs.

Thus, in some embodiments, “complementary” refers to a first nucleobase sequence that is at least 60%, 65%, 70%, 75%, 80%, 85%, 90%, 95%, 97%, 98%, or 99% identical to the complement of a second nucleobase sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, or more nucleobases, or that the two sequences hybridize under stringent hybridization conditions. “Fully complementary” means each nucleobase of a first nucleic acid is capable of pairing with each nucleobase at a corresponding position in a second nucleic acid. For example, in certain embodiments, an oligonucleotide wherein each nucleobase has complementarity to a nucleic acid has a nucleobase sequence that is identical to the complement of the nucleic acid over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, or more nucleobases.

### 2. SINGLE-STRANDED DNA ADAPTERS FOR QRRS

Embodiments of the present disclosure include compositions and methods pertaining to a quantitative next-generation sequencing (NGS) library preparation protocol and method for the optimization of sequencing quality and yield. In particular, the present disclosure provides a novel sequencing platform referred to as OmeSeq, which enables high-fidelity, dosage-sensitive genotyping and strain-level metagenomic profiling of various DNA and RNA templates across animal, plant, microbial, and viral genomes.

In accordance with these embodiments, the present disclosure provides forward and reverse single-stranded DNA (ssDNA) adapter molecules for use in performing a sequencing reaction (e.g., OmeSeq). The features of OmeSeq as described further herein are applicable to many sequencing platforms and methodologies, including but not limited to, reduced representation sequencing (RRS), shotgun whole genome and metagenome sequencing, full-length and partial cDNA sequencing of transcriptomes and meta-transcriptomes, and other specialized applications such as methylome sequencing. In some embodiments, quantitative RRS (qRRS) methods and compositions described herein can be used for high-throughput genome-wide marker genotyping, metagenome profiling, high-throughput in solution array-based targeted-assays (OmeSeq-Array), and low-through targeted-assays (OmeSeq-noSeq). Regardless of the sequencing application or platform, the ssDNA adapters of the present disclosure are a fundamental aspect of OmeSeq. In some embodiments, the ssDNA adapters include a probe binding region at the 5′ end of the adapters, a buffer region distal to the probe binding region, a barcode region distal to the buffer region, and a restriction enzyme overhang motif at the 3′ end of the adapters.

In some embodiments, the restriction enzyme overhang motif comprises a nucleic acid sequence complementary to an overhang sequence produced upon cleavage by a restriction enzyme. In some embodiments, the adapters are bound to a fragment of genomic DNA via complementation between the restriction enzyme motif of the ssDNA adapters and the genomic DNA produced upon cleavage by the restriction enzyme. In some embodiments, the restriction enzyme produced a 5′ overhang. In some embodiments, the restriction enzyme is NsiI or NlaIII, although any other suitable restriction enzyme can be used. In some embodiments, a single base change is included proximal to the overhang region and part of the restriction enzyme motif to ensure the restriction site is destroyed upon integration of adapter and genomic fragments.

In some embodiments, the ssDNA adapters also include a buffer sequence or region. The buffer region is based on unique sequences that ensure sequence diversity at each base position. The buffer sequence does not contain the restriction site described above in order to avoid digestion of adapter-genomic construct during secondary digest of any possible undigested or chimeric fragment. In some embodiments, the buffer region comprises a nucleic acid sequence from 4 to 8 base pairs in length. In some embodiments, the buffer region comprises a nucleic acid sequence from 5 to 7 base pairs in length. In some embodiments, the buffer region comprises a nucleic acid sequence that is 6 base pairs in length. In some embodiments, the buffer region is directly adjacent to the barcode region.

In some embodiments, the ssDNA adapters also include a barcode region. The barcode sequence or region does not contain the restriction site described above in order to avoid digestion of adapter-genomic construct during secondary digest of any possible undigested or chimeric fragment. In some embodiments, the barcode region is directly adjacent to the restriction enzyme motif. In some embodiments, the barcode region comprises a nucleic acid sequence from 5 to 12 base pairs in length. In some embodiments, the barcode region comprises a nucleic acid sequence from 6 to 11 base pairs in length. In some embodiments, the barcode region comprises a nucleic acid sequence from 7 to 10 base pairs in length.

In some embodiments, the ssDNA adapters also include a probe binding sequence or region. In some embodiments, the probe binding region facilitates binding to a substrate or probe. In some embodiments, the probe binding region facilitates binding to a separate nucleic acid molecule that is complementary to at least a portion of the nucleic acid sequence of the probe binding region. In some embodiments, the total length of the adaptor is from 25 to 100 base pairs. In some embodiments, the total length of the adaptor is from 30 to 90 base pairs. In some embodiments, the total length of the adaptor is from 35 to 80 base pairs. In some embodiments, the total length of the adaptor is from 40 to 70 base pairs. In some embodiments, the total length of the adaptor is from 45 to 60 base pairs.

Embodiments of the present disclosure also include a kit comprising the ssDNA adapters described above. In accordance with these embodiments, the kit can be used to perform a sequencing reaction or generate a nucleic acid library. In some embodiments, the kit also includes a compatible buffer for carrying out a molecular biology reaction (e.g., restriction digest), dNTPs (e.g., to facilitate nucleic acid synthesis or repair), a polymerase enzyme (e.g., to assemble RNA and/or DNA molecules), a restriction enzyme (e.g., to generate a 5′ overhang region), and/or cos-probes or pooled cos-probes.

Embodiments of the present disclosure also include a double-stranded genomic DNA fragment (e.g., genomic DNA) comprising the ssDNA adapter molecules described above appended to each end of the genomic DNA fragment. In accordance with these embodiments, the present disclosure also includes a composition comprising a plurality of genomic DNA fragments comprising the ssDNA adapter molecules described above.

The ssDNA adapters can also be used in accordance with OmeSeq as a solution-based array composition. In some embodiments, the array composition includes a plurality of DNA complementary overhanging sequence probes (“cos-probes”) capable of integration into targeted regions of a genomic template, and any of the ssDNA adapters described herein. For example, the cos-probes can be linked to the ssDNA adapters after integration of the cos-probe into a genomic template. In some embodiments, the cos-probes include at least one hairpin structure and an overhang complementary to the 5′ overhang of the restriction enzyme motif. In some embodiments, the cos-probes include a hairpin dsDNA with an overhang complementary to a NsiI overhang. The presence of the hairpin obviates the need to anneal a second strand in a separate step. This streamlines the processes of preparing the cos-probes.

In a particular embodiment of the present disclosure, OmeSeq can be used as a quantitative reduced representation sequencing (qRRS) platform. In accordance with these embodiments, the method can include appending the ssDNA adapter molecules of the present to a plurality of nucleic acid fragments digested to form a nucleic acid library. For example, the nucleic acid fragments can be fragments of genomic DNA that has been digested with a restriction enzyme. The ssDNA adapters can also be appended directly on to RNA or cDNA. The method also includes amplifying the plurality of nucleic acid fragments in the library using PCR and/or isothermal amplification. In some embodiments, appending the ssDNA adapter molecules comprises the use of cos-probes. The method also includes hybridizing the library to a nucleic acid sequencing platform and sequencing the genomic fragments. In some embodiments, the method obviates the need for performing a ligation reaction.

In some embodiments, the method results in at least 25% more sequencing reads. In some embodiments, the method results in at least 30% more sequencing reads. In some embodiments, the method results in at least 35% more sequencing reads. In some embodiments, the method results in at least 40% more sequencing reads. In some embodiments, the method results in at least 45% more sequencing reads. In some embodiments, the method results in at least 50% more sequencing reads. In some embodiments, the method results in 50% or more sequencing reads.

In some embodiments, the method comprises multiplexing. In some embodiments, the method removes chimeric fragments caused by reconstitution of restriction enzyme sites. In some embodiments, the method does not comprise PCR or ligation reactions. In some embodiments, the method minimizes barcode swapping. In some embodiments, the method enhances cluster generation. In some embodiments, the method comprises quantification of allele dosage in diploid and polyploid organisms. In some embodiments, the method comprises an error rate of less than 0.0002 across an entire length of a read, including proximal and distal ends that typically have high error rates.

In some embodiments of the method, the genomic DNA is obtained from one or more of bacteria, viruses, protozoa, plants, fungi, yeast, mammals, and any combination thereof. In some embodiments, the genomic DNA is obtained from a metagenome. In some embodiments, the genomic DNA is obtained from a microbiome. In some embodiments, the genomic DNA is obtained from an organism having a polyploid genotype.

### 3. COMPOSITIONS AND METHODS OF USE

ngsComposer: Fully Automated Pipeline that Includes Error Detection and Empirical-Based Next-Generation Sequencing Quality Filtering Algorithms.

Next-generation sequencing (NGS) is a widely applicable technology with over a decade of mainstream reception, yet ambiguity regarding best practices in data preprocessing remains. For example, Illumina short-read sequencing, the predominantly used platform due to its affordability and high yield, is considered the gold standard for NGS data quality. The sequence reads are often used to correct and improve data quality of sequences such as long reads derived from PacBio and Nanopore platforms. Nevertheless, Illumina short reads regularly contain sequencing errors that impact research (see, e.g., Glenn 2011, Goodwin et al. 2016). The detection of such errors from industry-derived metrics is often inflated and doesn't always account for elevated error rates at read ends. Even at low frequency, variant calling, de novo genome and transcript assembly, microbial profiling, and other sequence-based analytics are sensitive to sequencing errors (Fujimoto et al. 2010, Bokulich et al. 2013). For example, low frequency base calling errors can be misconstrued as minor alleles during variant or SNP calling and can result in false positives and false negatives during exact sequence matching-based strain identification.

Drawing biologically accurate inferences from NGS data requires high-quality sequence reads and each dataset requires ad hoc filtering based on desired application and library preparation technique. Trimming, demultiplexing, adapter removal, quality threshold filtering, artefact removal, and error-correction are common pre-processing steps. Multiple tools exist for each of these steps, each with its own approach and implementation. Quality-trimming of reads has been reported to have a profound impact on sequence assembly, SNP-calling, and gene expression. Demultiplexing tools often lack the ability to assign barcodes to pooled samples when a dual-barcoded library is used. Some tools lack the ability to handle variable length barcodes or barcode swapping, and misassignment of sample identities is an unresolved problem acknowledged by both independent research labs and Illumina (Kircher et al. 2011, Herten et al. 2015). Adapter removal algorithms vary in sensitivity and searching for variable barcodes in highly multiplexed libraries can be tedious. Error-correction methods assume a high-degree of sequencing depth (Yang et al. 2012). With each tool the number of individual parameters and the optimal sequential order of their application can significantly impact type I (false positive) and II (false negative) error rates. Additionally, many of the tools for processing short-read data are reliant on quality scores, which presents another possible source of discordance between methods.

Quality scores (Q scores) are a valuable metric for selecting high-quality reads. ASCII-encoded Q scores report the per-base probability of miscall based on optical fluorescence profiles measured as Q=−10 log 10(probability) (Ewing & Green 1998). Routinely, reads are processed based on Q scores to avoid inclusion of erroneously called bases. Over time, changes in Illumina sequencing platforms have shifted the interpretation of Q scores which affects the practicality and uniformity of their filtering performance (Minoche et al. 2011, Shin & Park 2016). Some platforms bin ranges of Q scores into classes due to differences in dye chemistry, surface reaction chemistry, or hardware data processing. The latest Illumina platform (NovaSeq 6000) has superior sequencing quality but only adopts 4 out of the standard 41 phred quality scores. In addition to instrumentation, Q scores are largely influenced by sample and library preparation (see, e.g., Fuller et al. 2009, Krueger et al. 2011, Pfeiffer et al. 2018). In the present disclosure, the ability to empirically improve quality filtering using known sequence motifs to parse reads independent of library preparation methods was investigated. As described herein, a universal set of best practices for empirical quality filtering is provided, including “ngsComposer,” a user-directed, fully automated, and modular pipeline prioritizing these best practices.

As would be recognized by one of ordinary skill in the art based on the present disclosure, there is a community need for improved tools in performing data preprocessing that makes few assumptions of Q scores and read composition, and instead relies on knowledge of library preparation and read sequence composition. Embodiments of the present disclosure provides metrics that highlight the efficacy of filtering reads using known sequence motifs coupled with, and in contrast with, Q score filtering. NGS reads from multiple Illumina platforms were measured for alignment accuracy and the fate of these reads under different filtering schemes, including optimal order of tools, was evaluated. Furthermore, a fully automated pipeline was developed that handles highly multiplexed data and enforces motif detection as a means of error detection and adapter removal.

Filtering NGS data is a complex but required task intended to retain accurately called reads. Customarily, Q scores are the exclusive determinant of the reliability of sequencing certainty. Q scores are a useful filtering metric. However, results described herein suggest that Q scores should not be taken at face value and can have variable interpretations across platforms. Underestimates of sequencing error in the barcode region described here imply that Q scores may not match the expected logarithmic Phred base-calling error probability. An inherent mechanism to know true read accuracy on a per-sequencing-run basis is difficult to conclude. Alignment to reference assemblies is possible with smaller genomes but may vary based on reference assembly and the significance of single base mutations may not be captured by alignment scoring penalties alone.

The pipeline described herein works off the assumption that reads containing an expected sequence near the 5′ end will contain higher quality base-calling across the entire read length and will contain fewer spurious mutations internal to the read body. Known sequence motifs originating from custom-designed adapters are the only known read portions in the resulting sequence reads, and therefore, it was expected that their fidelity would reflect read reliability to an extent. Emphasizing motif-filtering retains reliable base-calls of the quality required for SNP-calling and de novo assembly. Motif-detection is independent of platform, and, when used for data filtering, indirectly encompasses some of the errors that might be detected in other filtering approaches. Assessing the compression of non-unique reads uses a logic comparable to error correction methods, and results provided herein demonstrate that filtering of all types removes reads assumed to be unique as a result of sequencing error. Other marker-discovery pipelines such as TASSEL ignore Q score altogether when SNP-filtering due to a biasing tendency against SNPs on distal ends of reads (Glaubitz et al. 2013). Read reliability can be improved by implementing empirical motif-detection alongside threshold-filtering using machine-generated Q scores. Consideration to library preparation can improve overall sequencing reaction quality, and it gives researchers a practical framework to sort reads into populations.

Qmatey: A Versatile and Fully Automated Pipeline for Quantitative and Strain-Level Profiling of Metagenomes.

Metagenomics is the analysis of community sequencing data derived from environmental samples, facilitating the study of ecosystems. This methodology is essential for estimating diversity and abundance within microbiomes, which are communities of microorganisms found in various hosts and environments. Additionally, metagenomic analysis can uncover a microbiome's functional contribution to host health and productivity, establishing the importance of host-associated microbiomes in recent years (Berendsen et al. 2012, Miller et al. 2018, Adair et al. 2016, Mueller et al. 2019). The development of next generation sequencing technology steadily improves metagenomic techniques, which in turn necessitates the creation of accurate bioinformatic pipelines. Nevertheless, variability in metagenomic library preparation shapes downstream computational analysis.

Currently, two dominating methods of metagenomic library preparation are amplicon sequencing and shotgun metagenomic sequencing. Amplicon sequencing profiles the metagenome by targeting conserved markers such as the 16s rRNA gene for polymerase chain reaction (PCR) amplification, utilizing variable regions within the marker region for taxonomic classification. Computational analysis of amplicon libraries is database dependent. Sequences of high (97%) similarity are clustered into an operational taxonomic unit (OTU), which represents a classified microbial organism within a given sample. Bioinformatic pipelines such as QIIME 2 and MOTHUR process amplicon sequencing data with OTU clustering. Although amplicon sequencing analysis is elegant, computationally straightforward, and inexpensive, there are several limitations to amplicon library preparation that functionally limit metagenomic analysis.

For example, taxonomic classification is limited due to low taxonomic resolution and OTU misclassification while accurate microbial quantification is hindered due PCR bias and sequencing platform variability (Poretsky et al. 2014, Nguyen et al. 2016, Brooks et al. 2015, Zhou et al. 2011, Clooney et al. 201). Further, amplicon sequencing strategies are often focused on bacterial or fungal microorganisms, restricting the analysis of viral or higher-order eukaryotic DNA signatures (Boers et al. 2019). In contrast to amplicon sequencing, shotgun metagenomic sequencing attempts to holistically evaluate the metagenome without the use of marker-assisted amplicons, maximizing the amount of sequenced genomic material. This approach necessitates a variety of different computational algorithms for metagenomic analysis, broadly including de novo genome assembly and reference-dependent taxonomic profiling. Tools such as MetaPhlAn2, Kraken2, and HUManN2 integrate user-directed genome databases, which may vary from de novo assembled metagenomes to curated reference databases, to classify shotgun metagenomic reads. Although shotgun metagenomic sequencing heightens taxonomic resolution with strain-level classification, analysis is limited due to a lack of reproducibility associated with widespread computational algorithms and a lack of standardization (Doster et al. 2019, Sczyrba et al. 2017). Due to limitations in database curation, several shotgun metagenomic reads are not functionally diagnostic, which is why some bioinformatic pipelines utilize sample-specific pan-genome databases, yet it is clear the overabundance of non-diagnostic sequences interferes with functional metagenomic analysis and quantification (Nayfach et al. 2016). Additionally, the presence of eukaryotic paralogs and variability in genome size limits the accuracy of shotgun metagenomic sequencing (Beszteri et al. 2010).

In the present disclosure, limitations in metagenomic profiling and quantification were addressed by integrating a quantitative reduced representation sequencing (qRRS) strategy, an innovative library preparation method, with a novel bioinformatic pipeline called Qmatey (Quantitative Metagenomic Alignment and Taxonomic Exact matching). Qmatey is a modular, automated pipeline that includes reference-dependent normalization for abundance quantification, cross-reference database analysis for improved stringency, OTU clustering for amplicon sequencing, and exact-matching alignment for shotgun or reduced representation sequencing. By integrating both OTU clustering and exact matching algorithms into a modular format, embodiments of the present disclosure provide a robust pipeline capable of accurately profiling short-read metagenomic data regardless of library preparation. As provided herein, Qmatey's performance was validated through the analysis of metagenomic data collected from sweetpotato leaves with a qRRS strategy. Also, the Critical Assessment for Metagenomic Interpretation's (CAMI) open-source dataset was utilized to assess Qmatey's performance with shotgun metagenomic data.

Qmatey's modular workflow encourages robust metagenomic analysis, integrating NGS data regardless of library preparation method. These library preparation methods include 16S/ITS amplicon sequencing, shotgun sequencing and the more recently developed method, OmeSeq/qRRS (as described further herein). The latter captures genome-wide sequences/genes and is a similar power and resolution of detection as shotgun sequencing, at a cost lower than existing methods. The modular decision to profile metagenomes or microbiome with (i) OTU clustering for species to phylum-level profiling or (ii) exact-matching for strain-level profiling encourages researchers to choose the profiling algorithm best suited for their sequencing platform and library preparation strategy.

The utility of these profiling algorithms is further enhanced by an optional cross-reference filtering module, increasing the profiling stringency of the metagenomic data. By implementing these functions in a modular fashion, the user has significant control over profiling stringency, increasing taxonomic precision relative to accuracy or vice versa. In addition to Qmatey's modular profiling and filtering algorithms, Qmatey's novel reference normalization strategy facilitates accurate metagenomic quantification for researchers with spike-in standards and host-associated reference genomes.

The pipeline's pairwise correlation matrix, which requires a quantitative profile at strain level, shows promise for predicting inter-microbial interactions, identifying co-occurrence relationships across input samples. In the present disclosure, significant correlation values from the matrix showcases the utility validation for Qmatey's profiling accuracy using real qRRS data. By identifying confirmed multipartite interactions such as entomopathogenic and vector-viral relationships within the sweetpotato leaf microbiome, the correlation matrix and inference of the data provides validation of Qmatey's taxonomic profiling accuracy.

Genome-Wide Associations Detect Allele Dose-Dependent Metabolism and Transport Genes as Basis for Variation in Sweetpotato Storage Root Sugars.

Sweetpotato, Ipomoea batatas, is the seventh most important food crop in the world (CIP). The United States Department of Agriculture (USDA) reported a boost in sweetpotato cultivation of over 37% in their latest Agriculture Census report (USDA, 2019). Sweetpotatoes are rich in vitamins, minerals, and complex carbohydrates making them ideal for meeting the increasing demand from consumers for beneficial, nutritious vegetables. The United States accounts for less than 5% of global production but has seen an increase in domestic production with 30% of national supply sourced from North Carolina, an estimated value of US$55.7 million. Sweetpotato traits, such as the complex carbohydrates comprising the sugar profile, are sought after in marker assisted breeding programs. Determining the association between the traits and genes controlling trait expression, allows breeding programs to better maintain existing germplasms and curate new cultivars with desirable traits.

Genome wide association studies (GWAS) are performed to determine associations between traits of interest and potential genes driving observed phenotypic expression. In the last 15 years, GWAS studies have become more widespread in human health research. Relative to medical research, agricultural use of GWAS is new, having boomed in the last five years. The delay is due, in-part, to plants commonly being polyploids in contrast to diploid humans studied in medicine. In recent years, GWAS studies performed using genome-wide polymorphic DNA markers, are becoming important and effective methods for crop breeding programs. Genome-wide association studies can effectively detect quantitative trait loci (QTL) or target genes based on the association between genome-wide polymorphic markers and trait phenotypes.

Sweetpotatoes are a good example of a complex, polyploid plant in need of software that can meaningfully untangle their hexaploidy and highly heterozygous nature. Previous studies successfully designed an R software package, GWASpoly, that incorporates different models of gene action and investigates different types of kinships models for autopolyploids. These features address the requirements for polyploid GWAS studies to be effective. The other need for successful GWAS analyses is detection of genetic markers such as single nucleotide polymorphisms (SNPs) which are described in the present disclosure. GBSapp is a pipeline that integrates various software to detect thousands of SNPs allowing for a more robust genetic analysis (Wadl et. al. 2018).

Meaningful data has been generated by GWAS in Arabidopsis, maize, rice, wheat, tomato, and several other economically important crops. These studies have identified genes involved in abiotic and biotic stress response, plant growth and development, cell signaling, and inter- and intracellular transport to name a few. Exploration of candidate genes has led to progress in multiple marker assisted breeding programs. The elucidation of the genetic architecture controlling desirable traits frequently results in discoveries between major plant systems involved in phenotypic expressions.

Inter- and intracellular transport are vital plant processes directly linked to plant development, abiotic and biotic stress response, and nutrient transportation. Multiple genes encoding proteins involved in membrane channels with selectivity for cations transporting sugars; proteins involved in importing and exporting substrates involved in plant nutrition, growth, and stress response; and proteins that act as sugar transmembrane transporters have all been identified in Arabidopsis, maize, and/or rice. Existing annotation of genes involved in these plant processes are valuable tools when embarking on understanding the gene function in other economically important plants such as sweetpotatoes.

The economic importance of sweetpotato, internationally and nationally, is increasing along with consumer demands. The identification of candidate genes driving the sugar profile in sweetpotato provides meaningful guidance for marker assisted breeding programs. The traits driving flavor are controlled by several biological processes indicating high trait complexity. Dosage models for this polyploid crop indicate each trait has optimal allele amounts necessary to produce a crop with desirable trait. There is a robust presence of traits, and thus candidate genes, driving flavor in transport, abiotic and biotic stress response, and cell sensing/signaling.

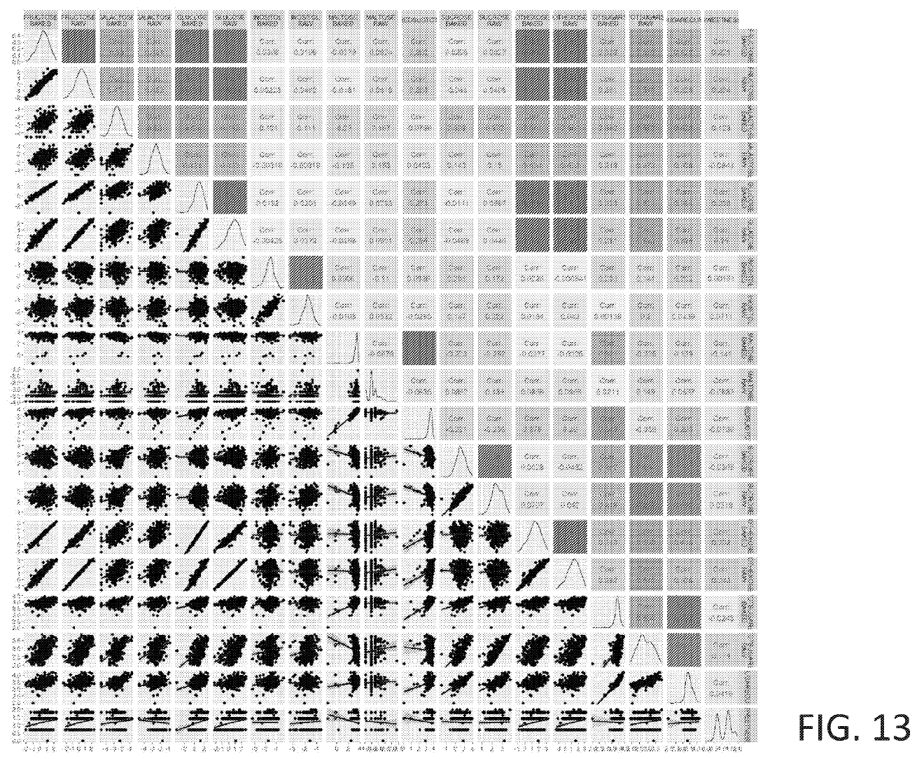

Of the eight traits with significant marker-trait associations investigated in the present disclosure, two traits have negative correlations with other sugar profile traits. Concentration of raw maltose has slightly negative correlations with concentration of raw galactose, total baked hexoses, and concentration of baked glucose and concentration of raw sugars has slightly negative correlations with total baked sugars. All other traits within the profile have slightly to 100% correlation (FIG. 13). Considering the traits within the profile are all sugars, it is expected to see the positive correlation amongst six of the eight traits.

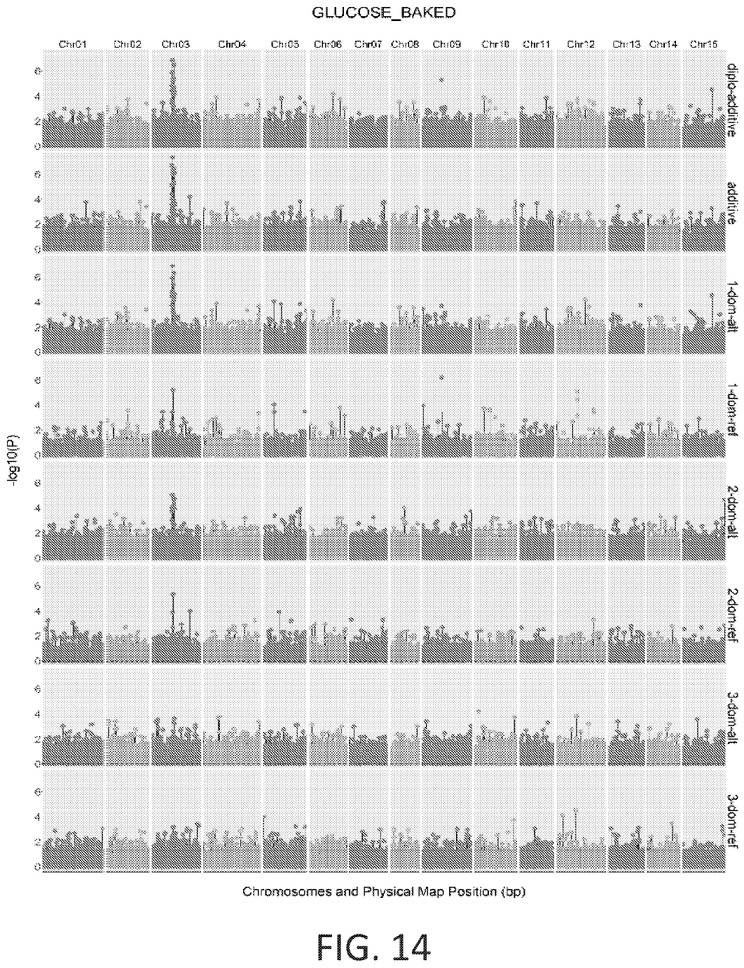

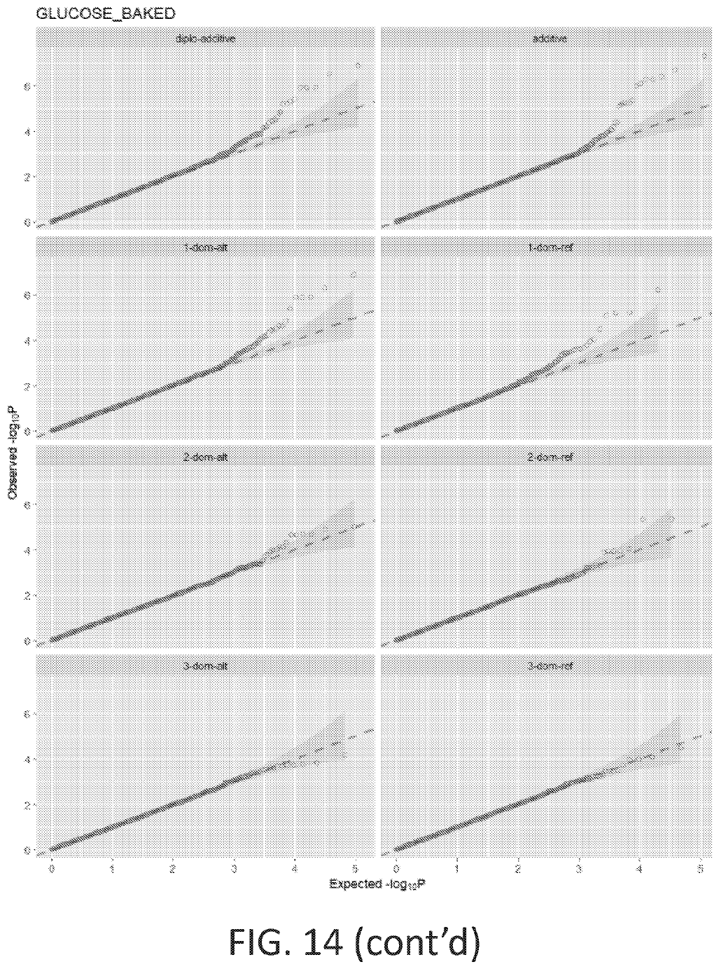

Half the traits with significant marker-trait associations are driven by three or more major plant processes (FIG. 14). Of those four traits, all have five or more candidate genes associated with trait expression (FIG. 14). The traits driving the sugar profile are controlled by several biological processes indicating high trait complexity. Intra- and intercellular transport and plant stress response are driving the sugar profile expression in six of the eight significant traits (FIG. 14). This suggests a meaningful connection between the significant traits driving the sugar profile and two major plant processes: Inter- and intracellular transport and plant stress response.

### 4. EXAMPLES

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

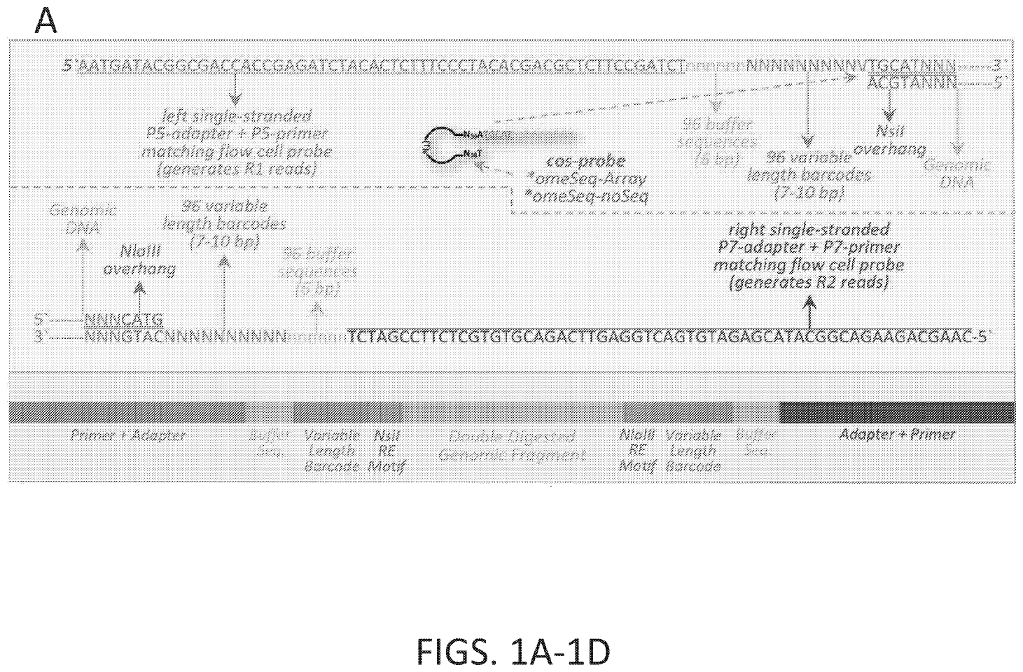

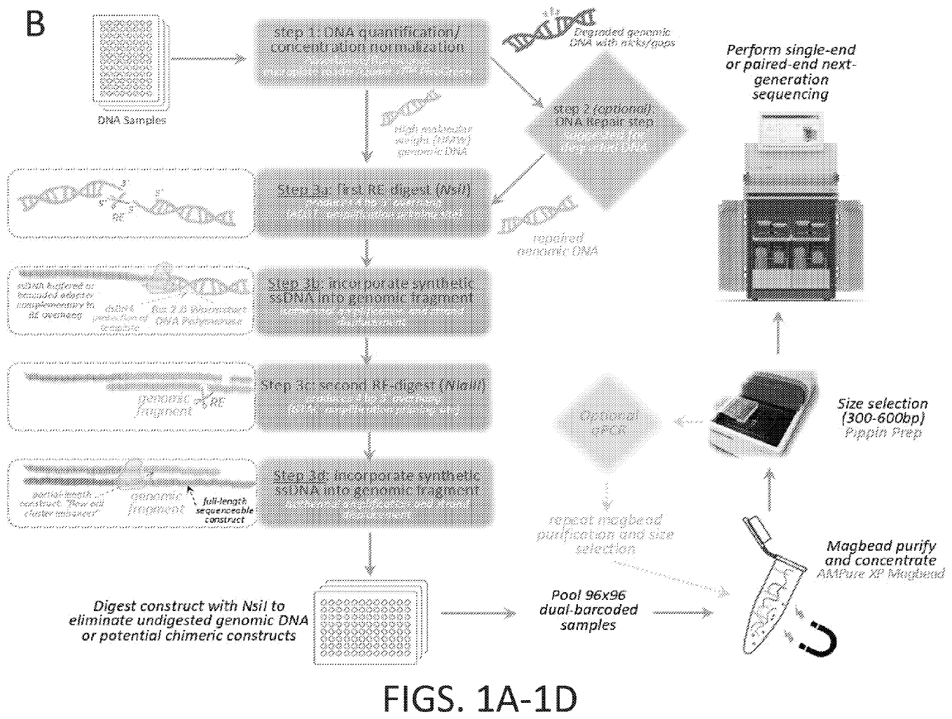

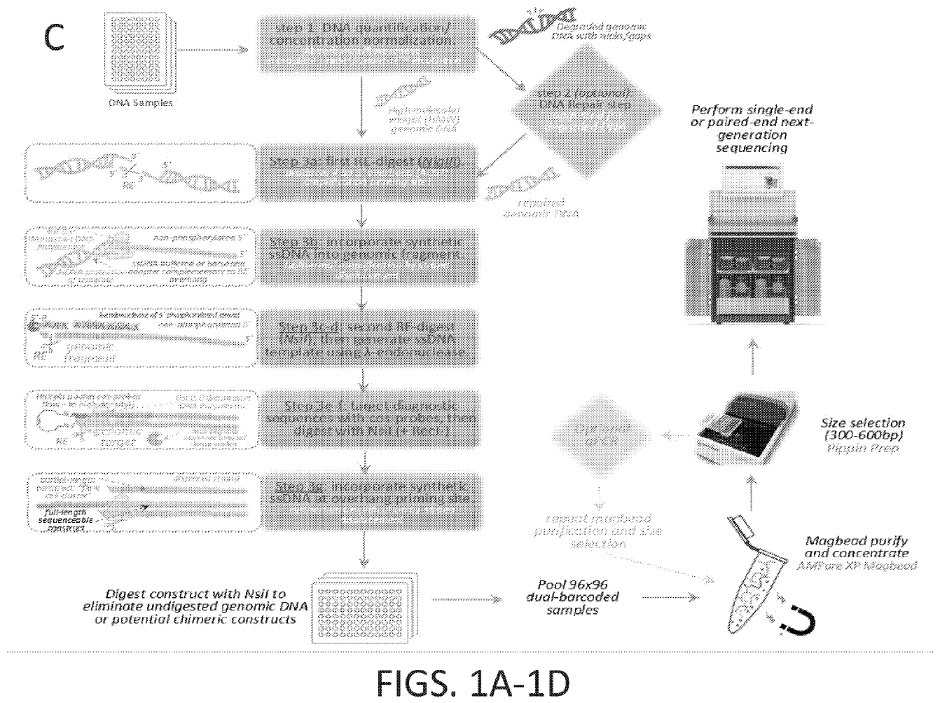

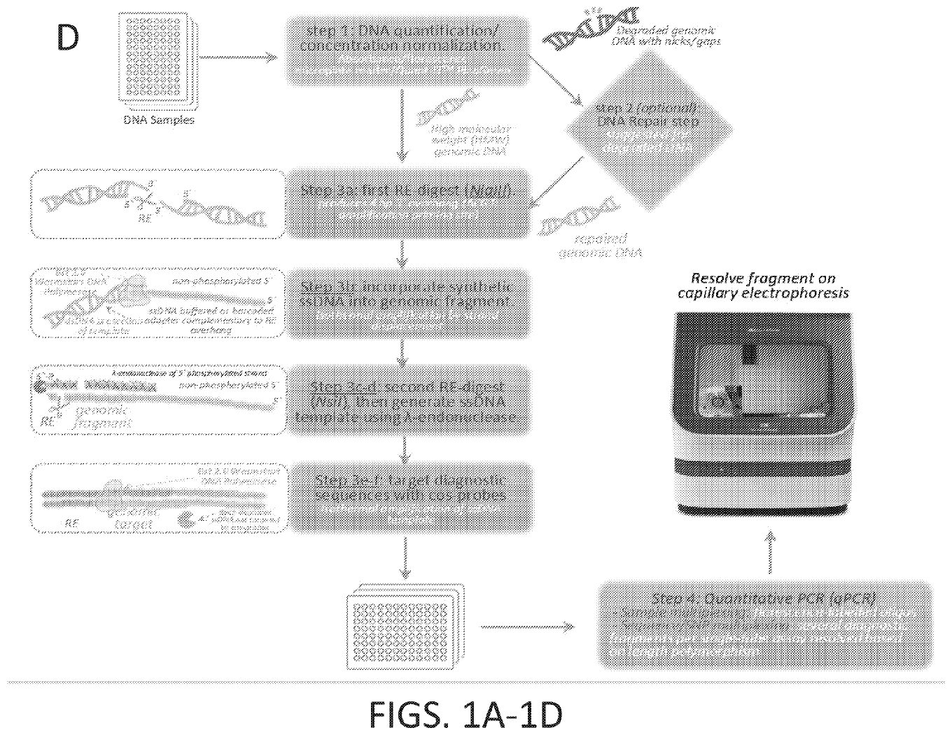

As shown in the representative schematic in FIG. 1A, the ssDNA adapters of the present disclosure (“buffered-barcoded adapter”) were designed for both single-end and paired-end short-read next-generation sequencing. A dual-barcoding of 96-x-96 adapters pairs allowed for a multiplexed assay of 9,216 pooled samples during paired-end sequencing (a 384-x-384 dual-barcoding allows for 147,546 pooled samples). The buffer sequence region ensures the variable length barcodes are shifted to a high-quality base calling region, which results in high-fidelity demultiplexing that minimizes barcode swapping. The buffered-barcoded adapters are engineered to completely eliminate chimeric fragments/constructs and lack these sequence motifs within them. The buffer and barcode regions are designed to account for substitution and indel error (based on Levenshtein/edit distance algorithm) and ensures nucleotide diversity required for optimal sequencing. A shift in assay design and component generates partial/incomplete constructs that enhance the percentage of cluster passing filter (e.g., an indication of signal purity).

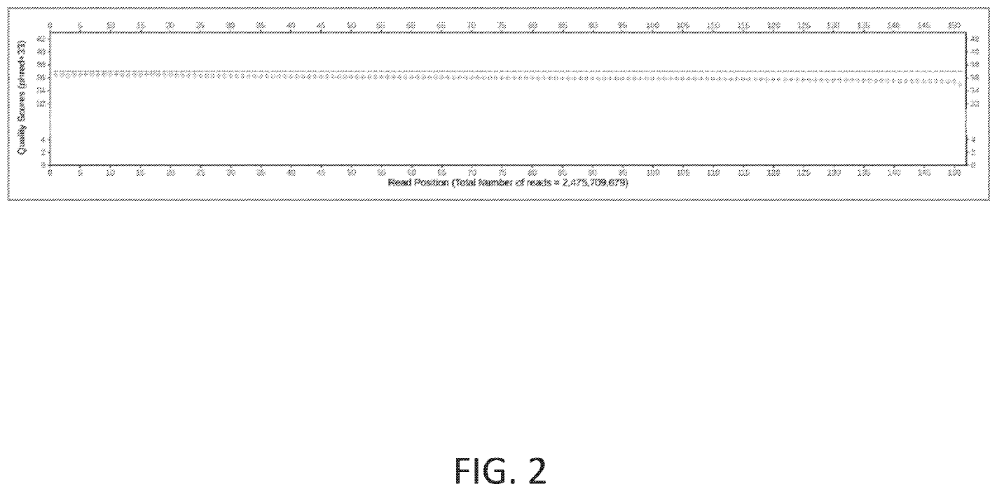

As shown in FIG. 2, consistent median “quality scores” were obtained at maximum on platform (Q37), including buffer and barcode sequences. The representative boxplot shows a blue dashed line as median, and an absence of boxes is indicative of minimal variation around median. Also, an absence whiskers indicates minimal/no outliers, and grey diamonds represent the mean. While the reads can still undergo quality filtering, this step is not necessary due to the optimized sequencing quality and yield. These methods increase yields due to the integration of the flow cell cluster enhancing strategy (e.g., Illumina maximum number of reads at 1.6 billion reads vs. OmeSeq's 55% more reads at 2.476 billion reads).

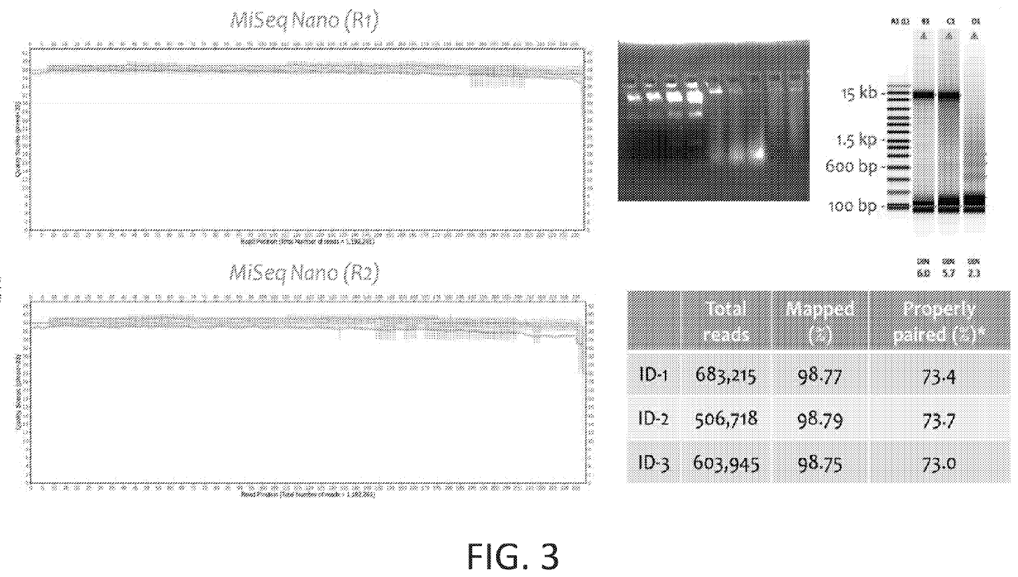

Additionally, FIG. 3 includes representative metrics showing the improved performance of OmeSeq/qRRS on highly degraded DNA samples that would normally fail clustering and sequencing using existing methods. The combination of a DNA repair step with OmeSeq delivered high-quality base calls, about 20% more yield, even representation of pooled samples independent of DNA quality, and the ability to map almost 99% of the reads to a draft reference genome.

### Example 2

An exemplary protocol using OmeSeq for quantitative genome-wide genotyping and quantitative strain-level metagenomic/microbiome profiling is provided below.

Samples were quantified (e.g., using Picogreen assay), and each sample was diluted to 20 ng/μl of DNA (with molecular grade water or low-EDTA TE buffer). Optionally, DNA with nicks and gaps was repaired (e.g., required for DNA samples that are highly degraded). Approximately 10 μl of DNA repair premix (Table 1) was added to 5 ul of 20 ng/μl (lower DNA concentrations can be used). Samples were incubated at 37° C. for 30 mins and then the enzymes were heat-killed with an incubation at 75° C. for 20 min. Samples were cooled at a rate of 20° C./min until reaching 21° C.

TABLE 1
DNA Repair Premix

| DNA repair premix | 1x vol. (ul) |
|---|---|
| Molecular grade ddH₂O | 6.1 |
| 10x CutSmart buffer | 1.5 |
| 10 mM ATP (final conc. 1 mM) | 1.0 |
| 10 mM dNTPs (final conc. 0.5 mM) | 0.5 |
| 50 mM NAD⁺ (final conc. 0.5 mM) | 0.1 |
| 10 mM DTT (final conc. 0.5 mM) | 0.5 |
| E. coli DNA polymerase I (0.1 U) | 0.1 |
| E. coli DNA ligase (0.1 U) | 0.1 |
| T4 polynucleotide kinase (0.1 U) | 0.1 |
| Total reaction vol. | 10 |

Restriction enzyme digests were performed, with each digest followed by isothermal amplification using the overhang as a binding site for the left and right adapters. Approximately 5 μl of NsiI-HF restriction enzyme digested premix (Table 2) was added to each DNA sample described above (assumed the 100 ng/μl DNA is in 1× CutSmart buffer). Samples were incubated for 1-3 hours at 37° C., and then the enzymes were heat killed 65° C. for 20 minutes.

To incorporate the forward/left single-stranded adapter at the NsiI 3′-overhang of the double-stranded DNA genomic fragment by strand-displacement, about 2.5 μl of 1 μM forward/left adapter-primer and 2.5 μl of isothermal amplification premix were added to digested DNA, incubated for 10 mins, and then heat killed at 80° C. for 20 mins. All 96 forward/left adapter were used to ensure nucleotide diversity (e.g., multiple adapters were used for each or specific samples if total number of samples were less than 96).

TABLE 2
NsiI-HF Restriction Enzyme and Isothermal Amplification Premix

| | 1x | | 1x |
|---|---|---|---|
| NsiI-HF restriction digest | vol. | Isothermal amplification | vol. |
| premix | (ul) | premix | (ul) |
| Molecular grade ddH₂O | 4.4 | Molecular grade ddH₂O | 1.25 |
| 10x CutSmart buffer | 0.5 | 10x CutSmart Buffer | 0.5 |
| NsiI (0.1 U) | 0.1 | 10 mM dNTPs | 0.5 |
| | | Bst 2.0 warmstart polymerase | 0.25 |
| Total reaction vol. | 5.0 | Total reaction Vol. | 2.5 |

About 5 μl NlaIII restriction enzyme digest premix (Table 3) was added to each sample above. Samples were incubated for 1-3 hours at 37° C., and then heat killed at 70° C. for 30 minutes.

To incorporate the reverse/right single-stranded adapters at the NlaIII 3′-overhang of the double-stranded DNA genomic fragment by strand-displacement, about 2.5 μl of 3 μM reverse/right adapter-primer and 2.5 μl of isothermal amplification premix (Table 3) were added to the digested DNA, incubated for 10 mins, and then heat killed at 80° C. for 20 mins. All 96 reverse/right adapter were be used to ensure nucleotide diversity (e.g., if experiments are based on only 96 samples, all 96 reverse/right adapters were combined and 2.5 μl aliquots were added μl to each sample).

TABLE 3
NlaIII-HF Restriction Enzyme and
Isothermal Amplification Premix

| | 1x | | 1x |
|---|---|---|---|
| NlaIII-HF restriction digest | vol. | Isothermal amplification | vol. |
| premix | (ul) | premix | (ul) |
| Molecular grade ddH₂O | 4.4 | Molecular grade ddH₂O | 1.25 |
| 10x CutSmart buffer | 0.5 | 10x CutSmart Buffer | 0.5 |
| NlaIII (0.1 U) | 0.1 | 10 mM dNTPs | 0.5 |
| | | Bst 2.0 warmstart polymerase | 0.25 |
| Total reaction vol. | 5.0 | Total reaction Vol. | 2.5 |

The library was pooled by combining aliquots of each sample into a single tube (e.g., 5 μl of each of the 96 samples to obtain 480 μl pool in a 1.5 ml tube.) Samples were digested with 2 μl of NsiI-HF for 1-3 hours to ensure elimination of undigested or chimeric fragment, then heat killed at 65° C. for 20 minutes.

To concentrate pooled samples and to eliminate small DNA fragments, a magbead purification was performed. About 723 μl (1.5× volume) of AMPure beads was added to the 482 μl pooled sample from above. Beads were then mixed. Samples were incubated for 5 mins at room temperature, and then placed on a magnetic stand to collect the beads. The supernatant was removed. The beads were then washed once with 500 μl of freshly made 70% ethanol while the sample tubes remained on the magnetic stand. The sample tubes were spun briefly in a centrifuge and the remaining 70% ethanol was removed. Samples were allowed to dry for 5 mins.

About 40 μl of TE buffer was added for elution. Sample tubes were from the magnetic stand and the beads were mixed. Sample tubes were placed back on magnetic stand and about 40 μl of clear solution comprising the DNA was removed.

Size selection was performed with Pippin Prep/BluePippin for the barcoded adapters (e.g., about 101-107 bp adapter sequences+200-450 bp target genomic insert), size selected between 300-600 bp fragments (or increments of 50 or 100 starting from 300 bp). Note that a maximum quantity of 10 μg of DNA per lane can be run on the Pippin Prep or BluePippin. Size-selected library can be checked with BioAnalyzer/TapeStation to ensure proper size selection. Sequencing reactions and/or PCR reactions can then be performed at this point, but are not required.

To incorporate sequences matching probes on the Illumina flow cell, the size-selected sample can be used for: (i) a PCR-free isothermal amplification for more accurate quantification, or (ii) a PCR amplification.

An exemplary protocol for PCR amplification of size selected library is provided below. A quantitative PCR reaction was performed by limiting number of PCR cycles to between 10 and 18.

TABLE 4
qPCR Reaction Mixture

| qPCR Mixture | 1x vol. (ul) |
|---|---|
| Library (10 ng/μl) | 10 |
| Phusion HF PCR masterMix (2x) | 12.5 |
| 10 uM Forward Primer (P2) | 1.25 |
| 10 uM Reverse Primer (P1) | 1.25 |
| Total reaction vol. | 25 |

PCR conditions: Samples were denatured at 95° C. (5 mins); 10-18 cycles were performed; samples were denatured at 95° C. for 15 sec; annealed at 65° C. for 30 sec; and extension at 72° C. for 60 sec. Final extension were performed at 72° C. for 5 min, and then held at 4° C. The library was then cleaned up after the PCR reaction by repeating the magbead purification and size selection (as described above). The library was quantified and size selection was confirmed using BioAnalyzer or Tapestation, and then diluted to 10 nmol/l in a total volume of 20 μl for sequencing (e.g., using an Illumina sequencer platform).

### Example 3

An exemplary in-solution OmeSeq-Array for targeted genotyping, quantitative strain-level metagenomic/microbiome profiling of endophytic communities, diagnostic assays is provided below.

Samples were quantified (e.g., using a Picogreen assay) and diluted to 20 ng/μl of DNA (with molecular grade water or low-EDTA TE buffer). Optionally, DNA with nicks and gaps was repaired (e.g., required for DNA samples that are highly degraded). About 10 μl of DNA repair premix (Table 1) was added to 5 μl of 20 ng/μl (lower concentrations of DNA can be used). Samples were incubated at 37° C. for 30 mins and heat killed at 75° C. for 20 min. Samples were cooled at a rate of 20° C./min until they reached 21° C.

TABLE 5
DNA Repair Premix

| DNA repair premix | 1x vol. (ul) |
|---|---|
| Molecular grade ddH₂O | 6.1 |
| 10x CutSmart buffer | 1.5 |
| 10 mM ATP (final conc. 1 mM) | 1.0 |
| 10 mM dNTPs (final conc. 0.5 mM) | 0.5 |
| 50 mM NAD⁺ (final conc. 0.5 mM) | 0.1 |
| 10 mM DTT (final conc. 0.5 mM) | 0.5 |
| E. coli DNA polymerase I (0.1 U) | 0.1 |
| E. coli DNA ligase (0.1 U) | 0.1 |
| T4 polynucleotide kinase (0.1 U) | 0.1 |
| Total vol. | 10 |

Restriction enzyme digests were performed, with each digest followed by isothermal amplification using the overhand as a binding site for the left and right adapters. For targeted sequencing, short sequence probes were used to target the NsiI 4 bp overhang (about an 8-18 bp distal sequence including the last base the diagnostic SNP position).

About 5 μl NlaIII restriction enzyme digest premix was added to each DNA sample from above (assumed the 100 ng/μl DNA is in 1× CutSmart buffer). Samples were incubated for 1-3 hours at 37° C., and then heat killed at 70° C. for 30 minutes.

The reverse/right single-stranded adapters were incorporated at the NlaIII 3′-overhang of the double-stranded DNA genomic fragment by strand-displacement by adding 2.5 μl of 3 μM reverse/right adapter-primer and 2.5 μl of isothermal amplification premix to digested DNA. Samples were incubated for 10 mins, and then heat killed at 80° C. for 20 mins. All 96 reverse/right adapter were used to ensure nucleotide diversity (e.g., multiple adapters were used for each sample if the total number of samples was less than 96). (The reverse/right single-stranded adapter was 5′ de-phosphorylated to avoid the degradation of the new synthesized strand by Lambda exonuclease.)

TABLE 6
NsiI-HF Restriction Enzyme and Isothermal Amplification Premix

| | 1x | | 1x |
|---|---|---|---|
| NsiI-HF restriction digest | vol. | Isothermal amplification | vol. |
| premix | (ul) | premix | (ul) |
| Molecular grade ddH₂O | 4.4 | Molecular grade ddH₂O | 1.25 |
| 10x CutSmart buffer | 0.5 | 10x CutSmart Buffer | 0.5 |
| NsiI (0.1 U) | 0.1 | 10 mM dNTPs | 0.5 |
| | | Bst 2.0 warmstart polymerase | 0.25 |
| Total reaction vol. | 5.0 | Total reaction Vol. | 2.5 |

About 5 μl NsiI-HF restriction enzyme digest premix (Table 6) was added to each sample from above. Samples were incubated for 1-3 hours at 37° C., and then heat killed at 80° C. for 20 minutes. To generate ssDNA genomic fragments with the reverse/right single-stranded adapter incorporated, 5 μl of Lambda exonuclease premix was added to each sample from above. Samples were incubated for 1-3 hours at 37° C., and then heat killed at 80° C. for 20 minutes.

TABLE 7
NsiI-HF Restriction Enzyme and Lambda Endonuclease Premix

| | 1x | | 1x |
|---|---|---|---|
| NsiI restriction digest | vol. | Lambda exonuclease | vol. |
| premix | (ul) | premix | (ul) |
| Molecular grade ddH₂O | 4.4 | Molecular grade ddH₂O | 4.3 |
| 10x CutSmart buffer | 0.5 | 10x CutSmart buffer | 0.5 |
| NsiI-HF (0.1 U) | 0.1 | Lambda exonuclease (0.1 U) | 0.2 |
| Total reaction vol. | 5.0 | Total reaction vol. | 5.0 |

To target genomic regions of interest with the cos-probe and incorporate the forward/left single-stranded adapter, 2.5 μl of 1 μM cos-probe and 2.5 μl of isothermal amplification premix (Table 2) were added to the ssDNA, incubated for 10 mins, and then heat killed at 80° C. for 20 mins.

About 5 μl NsiI-HF restriction enzyme digest premix (Table 3; included 0.1 U of RecJ_{f} endonuclease) was added to each sample from above. The overhang created by NsiI digestion provided a priming site for the ssDNA buffered and barcoded adapter, while the RecJ_{f} exonuclease degrades ssDNA genomic fragments that were not targeted by cos-probes. Samples were incubated for 1-3 hours at 37° C., and then heat killed at 65° C. for 20 minutes. To incorporate the forward/left single-stranded adapter at the NsiI 3′-overhang of the double-stranded DNA by strand-displacement, 2.5 μl of 1 μM forward/left adapter-primer and 2.5 μl of isothermal amplification premix (Table 2) were added to digested DNA from above, incubated for 10 mins, and then heat killed at 80° C. for 20 mins. All 96 forward/left adapter were used to ensure nucleotide diversity (e.g., multiple adapters were used for each specific sample if total number of samples were less than 96).

The library was pooled by combining aliquots of each sample into a single tube (e.g., 5 μl of each of the 96 samples to obtain a 480 μl pool in a 1.5 ml tube. Samples were digested with 2 μl of NsiI-HF for 1-3 hours to ensure elimination of undigested fragments, then heat killed at 65° C. for 20 minutes.

To concentrate pooled samples and to eliminate small DNA fragments, a magbead purification was performed. About 723 μl (1.5× volume) of AMPure beads was added to the 482 μl pooled sample from above, and the beads were mixed. Samples were incubated for 5 mins at room temperature, placed on a magnetic stand to collect the beads, and the supernatant was removed. Samples were washed once with 500 μl of freshly made 70% ethanol while the sample tubes remained on the magnetic stand. The sample tubes were spun briefly in a centrifuge and the remaining 70% ethanol was removed. Samples were allowed to dry for 5 mins. About 40 μl of TE buffer was added for elution. Sample tubes were removed from the magnetic stand, and the beads were mixed. Sample tubes were placed back on magnetic stand and about 40 μl of clear solution comprising the DNA was removed.

Size selection was performed with Pippin Prep or BluePippin to account for the barcoded adapters (e.g., 101-107 bp adapter sequences+200-450 bp target genomic insert), size selected between 300-600 bp fragments (or increments of 50 or 100 starting from 300 bp). A maximum quantity of 10 μg of DNA can be run per lane on the Pippin Prep or BluePippin. The library was checked with BioAnalyzer/TapeStation to ensure proper size selection. Sequencing reactions and/or PCR reactions can then be performed at this point, but are not required.

An exemplary protocol for PCR amplification of size selected library is provided below. A quantitative PCR reaction was performed by limiting number of PCR cycles to between 10 and 18.

TABLE 8
qPCR Reaction Mixture

| qPCR Mixture | 1x vol. (ul) |
|---|---|
| Library (10 ng/μl) | 10 |
| Phusion HF PCR masterMix (2x) | 12.5 |
| 10 uM Forward Primer (P2) | 1.25 |
| 10 uM Reverse Primer (P1) | 1.25 |
| Total reaction vol. | 25 |

PCR conditions: Samples were denatured at 95° C. (5 mins); 10-18 cycles were performed; samples were denatured at 95° C. for 15 sec; annealed at 65° C. for 30 sec; and extension at 72° C. for 60 sec. Final extension were performed at 72° C. for 5 min, and then held at 4° C.

The library was then cleaned up after the PCR reaction by repeating the magbead purification and size selection (as described above). The library was quantified and size selection was confirmed using BioAnalyzer or Tapestation, and then diluted to 10 nmol/l in a total volume of 20 μl for sequencing (e.g., using an Illumina sequencer platform).

### Example 4

An exemplary protocol using OmeSeq for low-throughput targeted genotyping, quantitative strain-level metagenomic/microbiome profiling of endophytic communities, and diagnostic assays is provided below.

Samples were quantified (e.g., using Picogreen assay) and each sample was diluted to 20 ng/μl of DNA (with molecular grade water or low-EDTA TE buffer). Optionally, DNA with nicks and gaps was repaired (e.g., optional but required for DNA samples that are highly degraded). About 10 μl of DNA repair premix (Table 1) was added to 5 μl of 20 ng/μl (lower concentrations of DNA can be used). Samples were incubated at 37° C. for 30 mins and heat killed at 75° C. for 20 min. Samples were cooled at a rate of 20° C./min until reaching 21° C.

TABLE 9
DNA Repair Premix

| DNA repair premix | 1x vol. (ul) |
|---|---|
| Molecular grade ddH₂O | 6.1 |
| 10x CutSmart buffer | 1.5 |
| 10 mM ATP (final conc. 1 mM) | 1.0 |
| 10 mM dNTPs (final conc. 0.5 mM) | 0.5 |
| 50 mM NAD⁺ (final conc. 0.5 mM) | 0.1 |
| 10 mM DTT (final conc. 0.5 mM) | 0.5 |
| E. coli DNA polymerase 1 (0.1 U) | 0.1 |
| E. coli DNA ligase (0.1 U) | 0.1 |
| T4 polynucleotide kinase (0.1 U) | 0.1 |
| Total reaction vol. | 10 |

Restriction enzyme digests were performed, with each digest followed by isothermal amplification using the overhang as a binding site for the fluorescently labelled forward/left cos-primer and reverse/right primers. The forward/left cos-primer targets the NsiI 4-bp overhang (about an 8-18 bp distal sequence including the last base at the diagnostic SNP position). The reverse/right primer binds the NlaIII 4-bp overhang. (Note: Illumina P5/P7 buffer sequences and barcodes are not required for this library.)

About 5 μl NlaIII restriction enzyme digest premix was added to each DNA sample from above (assumed the 100 ng/μl DNA is in 1× CutSmart buffer). Samples were incubated for 1-3 hours at 37° C., and then heat killed at 70° C. for 30 minutes.

The reverse/right adapter was incorporated at the NlaIII 3′-overhang of the double-stranded DNA genomic fragment by strand-displacement, 2.5 μl of 3 μM reverse/right primer and 2.5 μl of isothermal amplification premix were added to digested DNA from, incubated for 10 mins, and then heat killed at 80° C. for 20 mins. (The reverse/right single-stranded adapter was 5′ de-phosphorylated to avoid the degradation of the new synthesized strand by Lambda exonuclease.)

TABLE 10
NlaIII-HF Restriction Enzyme and
Isothermal Amplification Premix

| | 1x | | 1x |
|---|---|---|---|
| NlaIII-HF restriction digest | vol. | Isothermal amplification | vol. |
| premix | (ul) | premix | (ul) |
| Molecular grade ddH₂O | 4.4 | Molecular grade ddH₂O | 1.25 |
| 10x CutSmart buffer | 0.5 | 10x CutSmart Buffer | 0.5 |
| NlaIII (0.1 U) | 0.1 | 10 mM dNTPs | 0.5 |
| | | Bst 2.0 warmstart polymerase | 0.25 |
| Total reaction vol. | 5.0 | Total reaction Vol. | 2.5 |

About 5 μl NlaIII-HF restriction enzyme digest premix was added to each sample from above. Samples were incubated for 1-3 hours at 37° C., and then heat killed at 80° C. for 20 minutes. To generate ssDNA genomic fragment with the reverse/right single-stranded adapter incorporated, 5 μl of Lambda exonuclease premix was added to each sample from above. Samples were incubated for 1-3 hours at 37° C., and then heat killed at 80° C. for 20 minutes.

TABLE 11
NsiI-HF Restriction Enzyme and Lambda Endonuclease Premix

| | 1x | | 1x |
|---|---|---|---|
| NsiI restriction digest | vol. | Lambda exonuclease | vol. |
| premix | (ul) | premix | (ul) |
| Molecular grade ddH₂O | 4.4 | Molecular grade ddH₂O | 4.3 |
| 10x CutSmart buffer | 0.5 | 10x CutSmart buffer | 0.5 |
| NsiI-HF (0.1 U) | 0.1 | Lambda exonuclease (0.1 U) | 0.2 |
| Total reaction vol. | 5.0 | Total reaction vol. | 5.0 |

To target genomic regions of interest with the cos-probe (hairpin and ssDNA buffer/barcoded adapters not required for OmeSeq-noSeq), 2.5 μl of 1 μM cos-primer and 2.5 μl of isothermal amplification premix (Table 2) were added to the ssDNA genomic template from above, incubated for 10 mins, and then heat killed at 80° C. for 20 mins.

The library was pooled by combining aliquots of each sample into a single tube. Samples were digested with 2 μl of NsiI-HF for 1-3 hours to ensure elimination of undigested fragments, then heat killed at 65° C. for 20 minutes.

Constructs in the library can be resolved using capillary gel electrophoresis. The florescent labeling allows differentiating allelic variants between fragments from the same locus, while fragment lengths allow for multiplexing target sequences/SNPs in a single tube assay (50-plex to a few hundred-plex). Additional sample multiplexing (2-plex) can be achieved using a 4 florescent dye system. Capillary gel electrophoresis platforms having multiple capillaries allow for running multiple samples on a single machine in a single run (e.g., 96- or 384-plex on ABI prism). Since it is a quantitative assay, the electropherogram peaks can be used to estimate allele dosage. Alternatively or additionally, instead of electrophoresis-based assays, an inexpensive low-throughput array can be used for the assay. In this case, the cos-primer will be used as cos-probe fixed to a silicon-based chip and a reaction like the one described above will be employed for the assay. Like SNP chips and micro-arrays, the assay will be florescence-based.

### Example 5

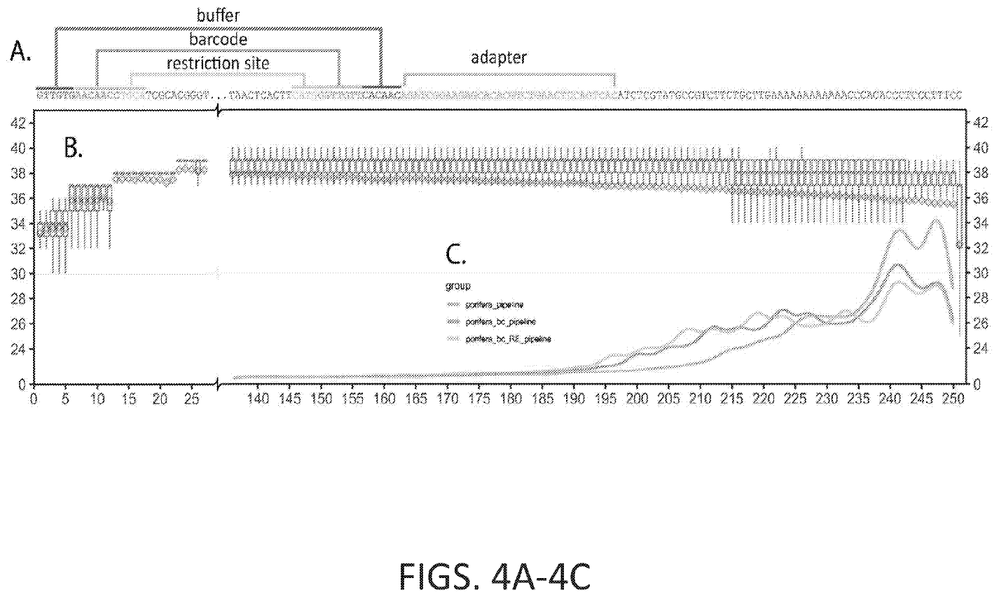

Library Design and Empirical Q Score Assessment. To assess the effectiveness of Q scores against motif-detection approaches, NGS data from sweetpotato (Ipomoea batatas) were considered from multiple Illumina platforms, each with distinct profiles. Fastq reads originating from Miseq, HiSeq 2500 Rapid Run, HiSeq 2500 High Output, and NovaSeq 6000 represent a variety of read length, dye chemistry, and various levels of Q score binning. All DNA libraries were prepared using custom-designed adapters and enzyme fragmentation (e.g., high- and low-quality sequence data. The latter is based on sub-optimal protocol parameters, which allows for the production of low quality base calls in order to highlight the strength of specific algorithms for error detection and filtering). Each of the adapter pairs (dual-barcoded library) include a 6 bp buffer region, 7-10 variable length barcode sequence, and a 4 bp motif complementary to each of the two restriction cut sites in the insert DNA (FIG. 4A). Combinations of the 96 left and 96 right adapters provide a 9,216-multiplex level. By using adapters that contain a fixed-length and high-diversity “buffer sequence” region (as implemented in OmeSeq/qRRS protocol), the base calling is allowed to stabilize before base calling in the barcode regions starts (Mitra et al. 2015). This has a protective effect on the barcode sequences used to determine sample identity, as the initial bases in a sequencing by synthesis reaction tend to harbor lower Q scores (FIG. 4B). Basic end-trimming was performed by ‘scallop.py’ in this buffer region before demultiplexing.

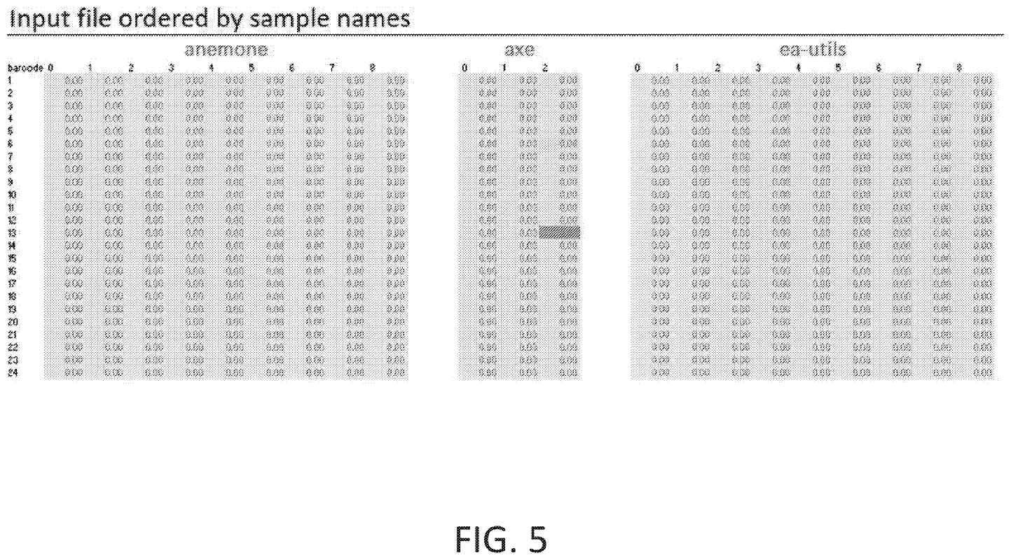

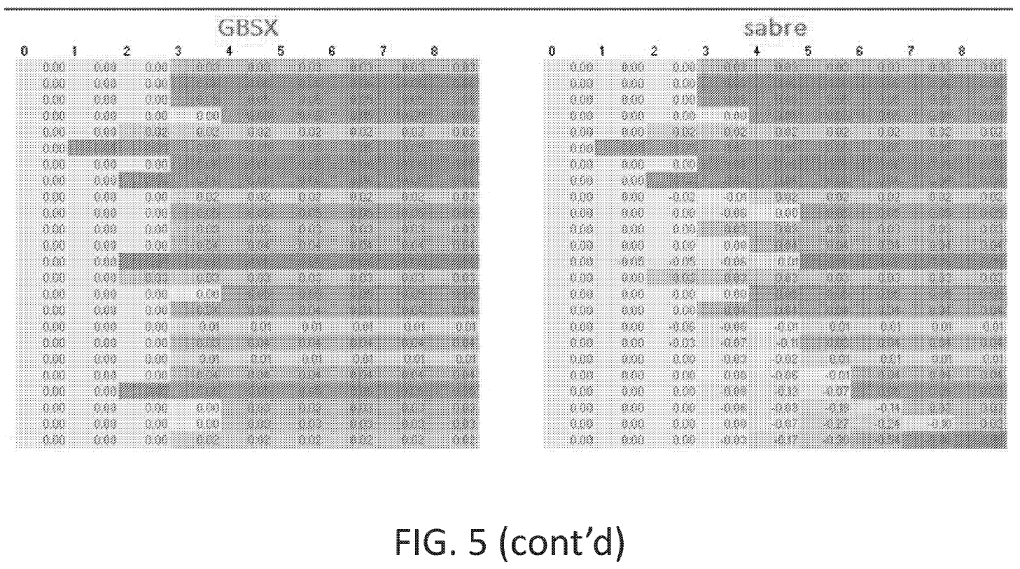

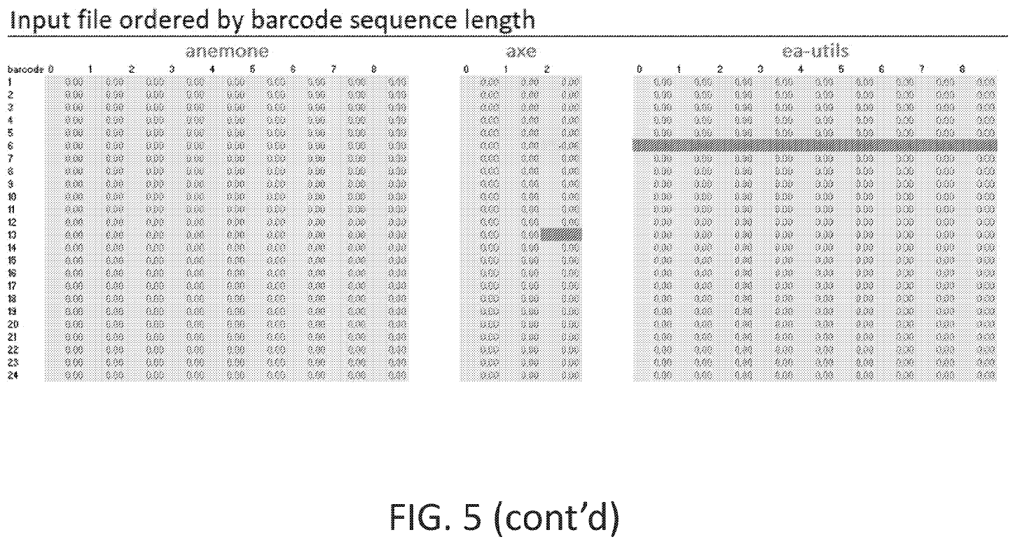

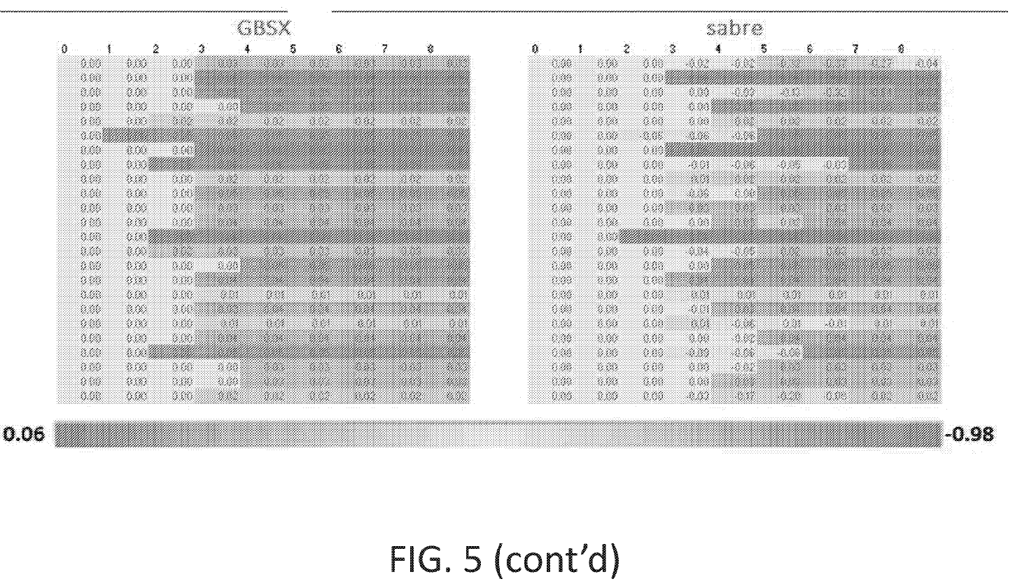

Variable length barcodes with a minimum Levenshtein/edit distance of 3 were implemented to prevent platform-derived phasing error. The tool ‘anemone.py’ was used to demultiplex the libraries using a dual-indexed barcoding scheme, which is known to improve assignment accuracy. Anemone.py prevents sample misassignment in the event that multiple barcodes have equal Hamming distance from a given read. Reads that align to multiple barcodes remain unassigned. Misassignment can be found in several widely used demultiplexing tools, which apply sample ids preferentially to the first barcode identified when “nesting” occurs (FIG. 5).

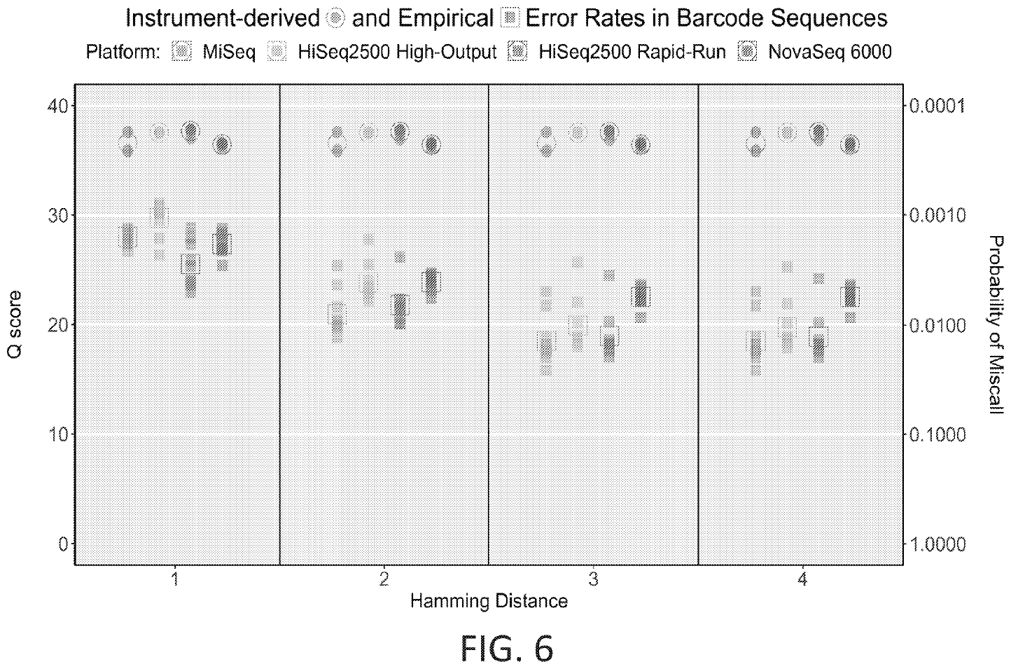

Taking advantage of anemone.py's false positive sensitivity, the sequencing accuracy of barcode regions was tested by demultiplexing at increasing levels of hamming distances. The per-base rate of mutation was calculated alongside the reported per-base Q scores (FIG. 6). Calculated miscall probabilities were lower than those produced by the sequencing hardware with mean differences across platforms between 9 and 17, corresponding with approximately 8 to 50 times increased probability of miscall.

### Example 6

Evaluating Read Populations Using Expected Motifs. Read depth refers to the frequency of reads aligning uniquely to a given reference locus. The restriction enzyme-based reduced representation sequencing libraries examined in the present disclosure consist of numerous, non-overlapping fragments of DNA that align flush with one another. Collapsing instances of 100% identical sequence reads with a NGS dataset, termed the genome-wide compression rate (or simply the compression rate), was used as a proxy for unbiased error rate estimates The compression rate value is an approximation of the average read depth across the genome (e.g., number of times an allele was sequenced). High error rates due to base mutations will increase the generation of new novel reads and consequently low compression rates.

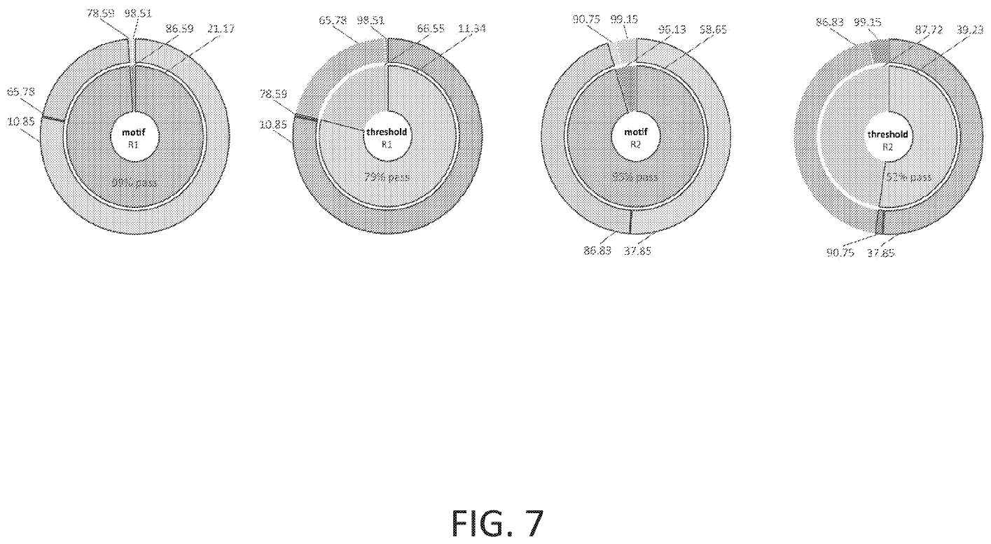

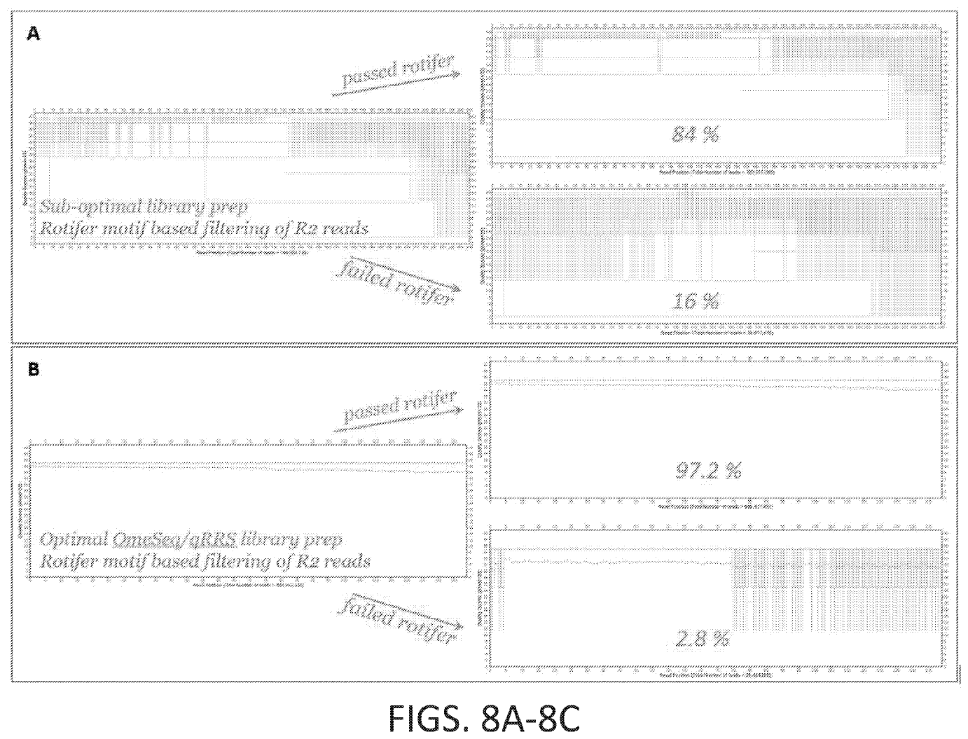

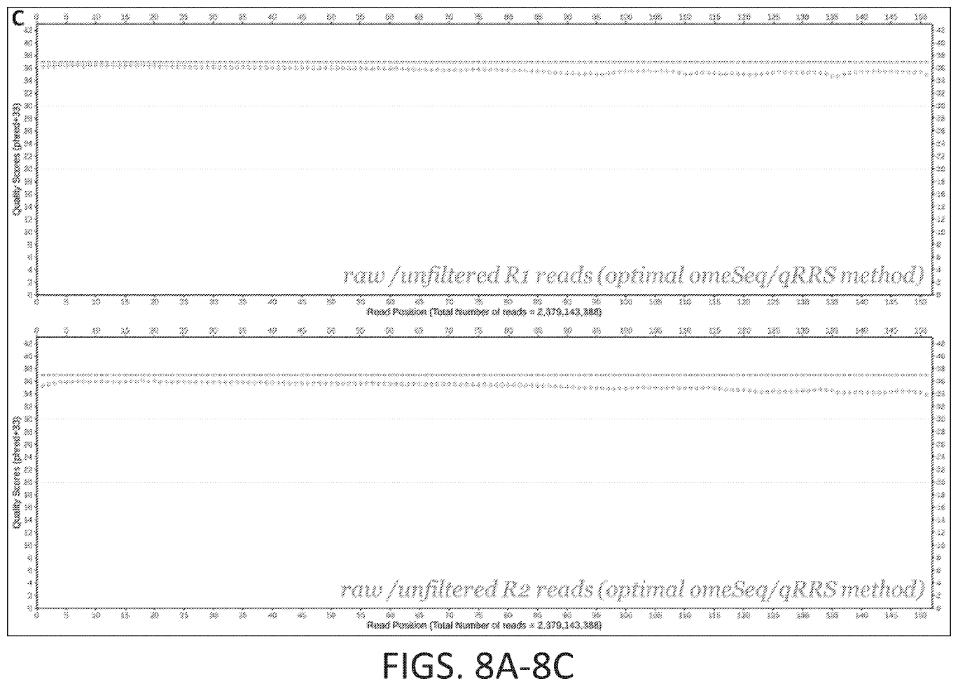

After barcode removal, error-free restriction enzyme cut sites were used as an early indicator of read reliability (motif-based filtering). Reads with error in the motifs were observed to also contain higher error rates along the entire length of the read compared to intact motifs (FIG. 8). Reads filtered using sequential application of classic Q score threshold-filtering and motif-filtering produced populations of passing and failing reads for the cross section of both approaches. A higher false negative rate was expected for stringent Q score threshold filtering and a high false negative rate for relaxed Q score threshold filtering. Since the threshold is very subjective, there is currently no best practice for the optimal threshold to minimize these false positive and false negative rates. The motif-based filtering approach, which is not subjective, complements the Q score threshold-filtering approach. In all datasets analyzed across all NGS sequencing platforms, the highest compression was achieved when both filtering approaches were applied (FIG. 7). Reads failing these filtering approaches consisted of more unique reads (due to increased error rates) and consequently had a lower compression rate.

### Example 7

Improved Adapter Detection Using Barcode-specific Search Schema. Adapter removal is an important step in many NGS libraries and detection can be improved using expected motifs. Adapter sequences including barcode and restriction site motifs are expected to increase adapter sensitivity as these sequences are further upstream of the characteristic 3′ drop in sequence quality. Previous work has shown the inclusion of restriction motifs improved adapter detection. As described herein, the tool porifera.py has been developed to k-mer walk through a list of adapters and search a user-defined number of rounds until aligned k-mers point to the same start index or a read is deemed to be adapter-free. The k-mer approach avoids local alignment issues encountered when a string of “A” or “G” sequencing artifacts appear in the instance of a deeply embedded adapter. The ngsComposer pipeline mode narrows the adapter search space by only attempting to align reads with their associated adapters which contain sample-specific barcodes. Adapters including barcodes and barcodes with restriction sites detected reads at a higher frequency and removed non-genomic DNA in the miseq dataset (FIG. 4C).

As provided herein, NgsComposer is designed with simplified user input at every critical step in data filtering. Any of the provided tools may be run individually or as an automated pipeline. In pipeline mode, users have the option to see read summaries and qc plots and reissue variables on the fly as a part of “walkthrough” mode. Multiple libraries from different sequencing runs may be combined together, each with its own set of barcodes. In pipeline mode, paired end reads are automatically recognized and pairing preserved throughout. Reads that become uncoupled due to partner removal are retained in all subsequent steps in a single end reads directory.

### Example 8

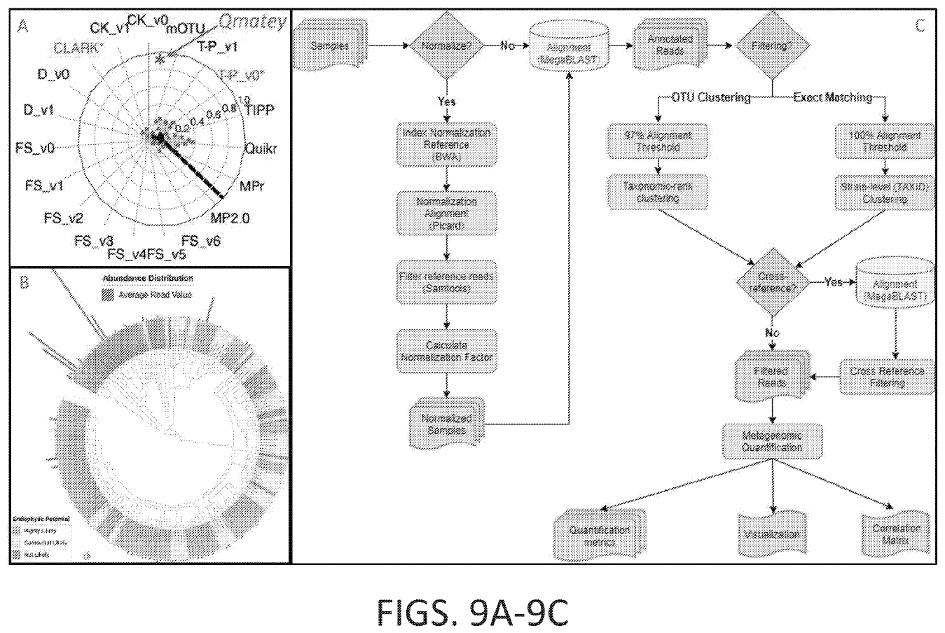

Metagenomic Validation and Composition of the Sweetpotato Diversity Population. In combination with a reduced representation sequencing strategy, Qmatey quantifies a biologically relevant taxonomic profile of the sweetpotato leaf microbiome. Regardless of limited sequencing depth with only 5.02% metagenomic coverage, Qmatey filtered approximately 1,951 significant strains and species from the Diversity population. Strains and species (n=199) present within 5% of the individuals were evaluated (FIG. 9B). Because there is no universally accepted standard for reduced representation metagenomic profiling, the validity of the taxonomic matches was evaluated by cross-referencing relevant literature. Of the 87 genera present within the profile, 25 are confirmed to be on, in, and around sweetpotato or sweetpotato-associated insects such as Bemisia tabaci. About 35 genera have not been confirmed as sweetpotato-associated genera but represent putatively novel matches that are present within similar crops and insects. Lastly, 27 genera contain taxonomic matches unlikely to be associated with the sweetpotato leaf microbiome, indicating error in community classification or database curation. Some sources of classification error are clear. Taxonomic matches associated with Ipomea and other Virdiplantae are potentially sweetpotato reads that were not filtered out of the host-reference genome alignment due to an incomplete genome assembly.

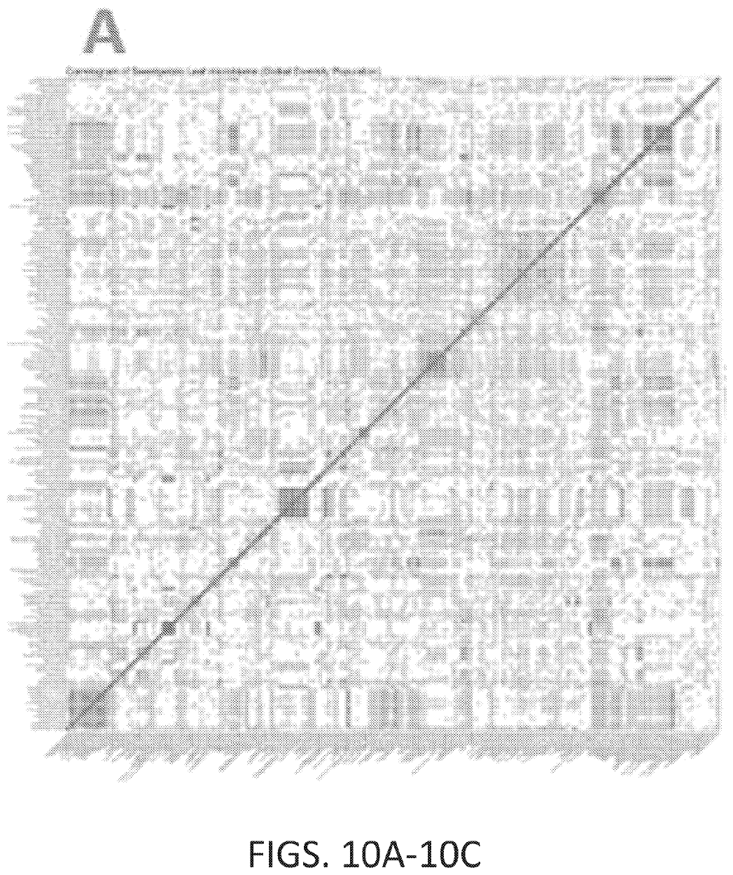

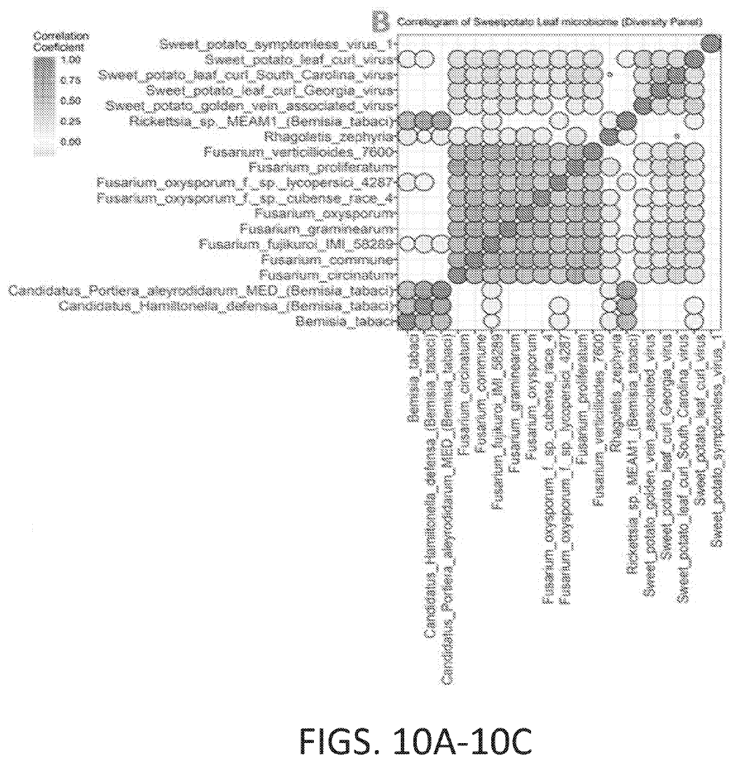

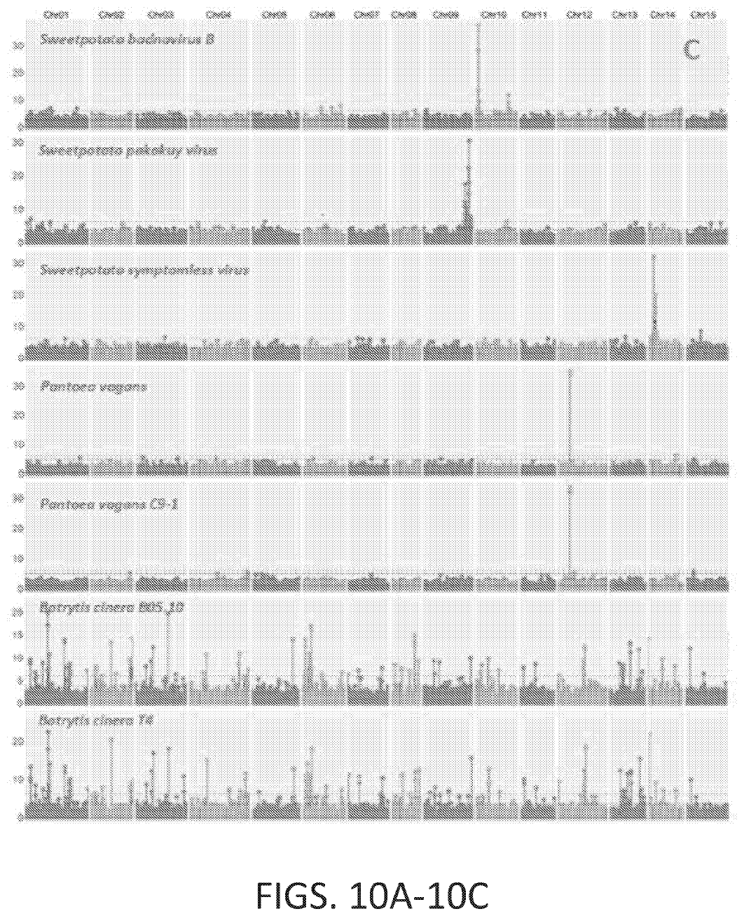

Although a few of the most abundant matches represent erroneous artifacts of the sweetpotato genome, other highly abundant matches are further confirmed through pair-wise inter-microbial correlations. For example, most species and strains within the Fusarium genus are negatively correlated with Rhagoletis zephyria (p<0.05, FIG. 10). This negative interaction indicates a potential relationship between Fusarium and Rhagoletis within the diversity population, which is supported by Fusarium's entomopathogenic potential. Additionally, sweetpotato whitefly (Bemisia tabaci) is positively correlated with confirmed endophytic bacteria and sweetpotato viruses (p<0.05, FIG. 10). Three of the high abundance sweetpotato viruses are significantly correlated with their vectors, providing additional support for the legitimacy of the taxonomic matches of the highly abundant viral taxa.

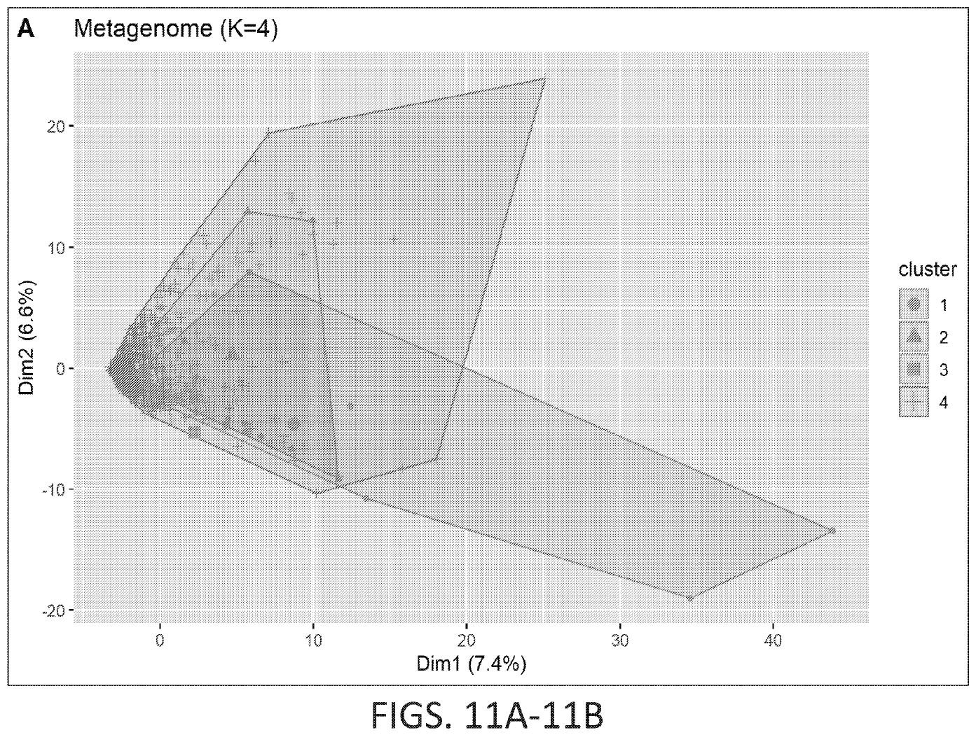

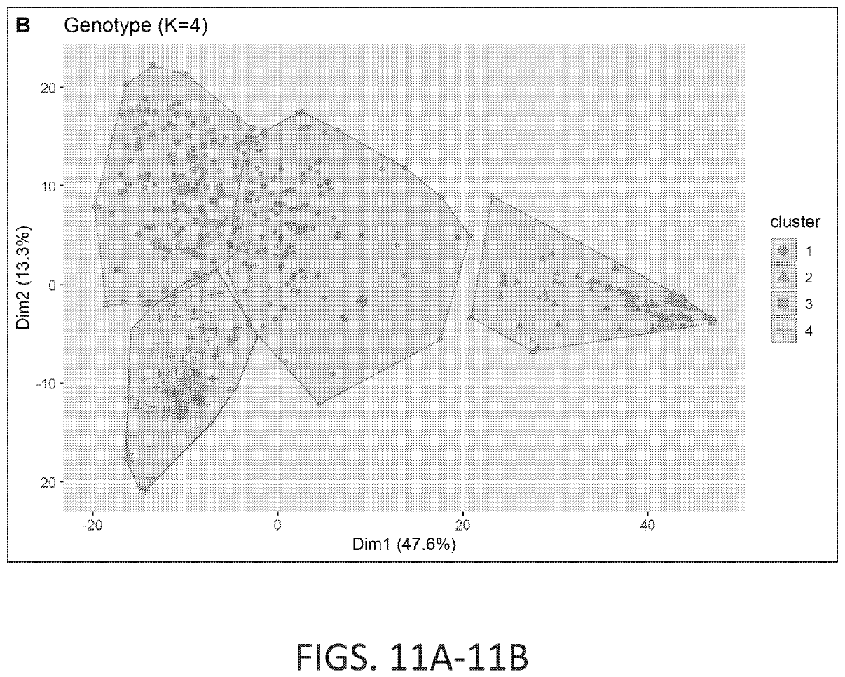

In addition to validating the taxonomic profile of the population's metagenome, variation in metagenomic composition within the population was examined with a machine-learning, K-means clustering approach (FIG. 11A). The metagenomic composition within the population is homogenous, suggesting the consistency of metagenomic taxa throughout the population regardless of individual dissimilarity. To evaluate genomic variation within the sweetpotato population, this clustering algorithm is applied to the population's additive kinship matrix, estimating the relatedness of individuals (FIG. 11B). The K-means clustering approach on the kinship matrix identifies genomic similarity and dissimilarity within the population, constructing four groups (or sub-populations) of relative ancestral relatedness. Clustering pattern reveals that individual host-microbiome composition is driven by a genetic architecture independent of shared ancestry within sub-populations. Clustering pattern reveals that individual host-microbiome composition is driven by a genetic architecture that is independent of shared ancestry within sub-populations. These suggests that variation in recruitment of the leaf microbiomes for most part is not driven environmental factors or spatial and geographical distribution of accessions. This agrees with explanations about how the niche in the leaf might be tightly controlled by physical and chemical properties of the leaf. Clustering of accessions based on microbiome composition suggest recruitment of some member microbes might be evolutionarily conserved based on fine-tuned molecular interactions stabilized by host-microbe co-evolution. Nevertheless, variation exist within some accessions that diverge from this consensus (FIG. 11A).

### Example 9

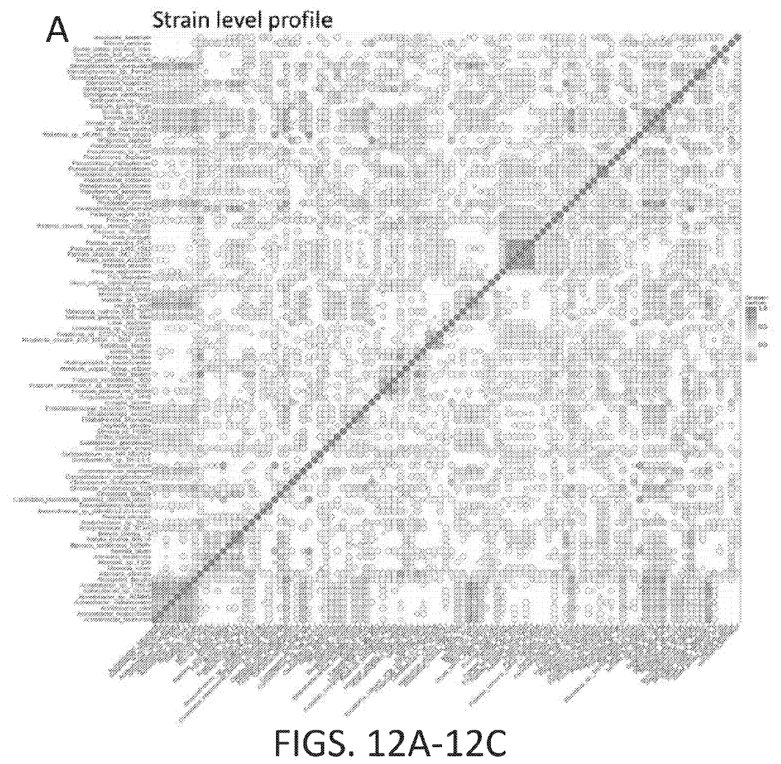

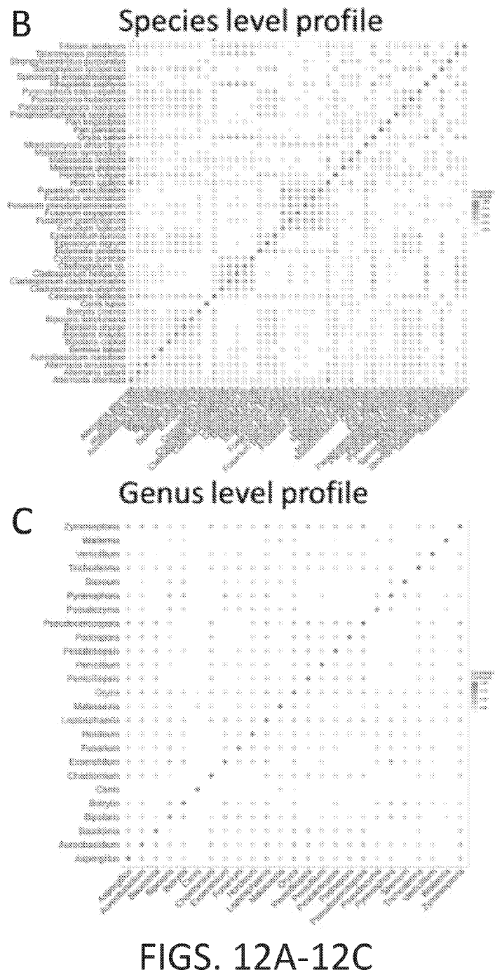

Qmatey calculates pairwise correlation values to analyze the co-occurrence of taxa within the metagenomic profile and across the sweetpotato germplasm. A negative correlation coefficient (red) indicates an antagonistic interaction between microbes (e.g., competition, and anti-microbial production), while a positive correlation coefficient (blue), indicates synergistic interactions or co-occurrence/co-evolution that have stabilized between microbes and the plant host. Qmatey's statistically significant pairwise correlation value does not imply causal interaction, but it provides a framework for specifically evaluating putative, inter-microbial interactions within the metagenome. For the strain level profile, Qmatey's pairwise correlation matrix yielded 13,459 correlation values greater than zero and 2,420 correlation values less than zero (p<0.05, FIG. 12A). Because Qmatey's or other tools' OTU algorithm clusters sequences into a representative taxonomic unit of interest, the number of observations is effectively reduced above strain-level. Consequently, the strain-level profile has several orders of magnitude more statistically significant correlation values compared to the combined correlation values of the species, genus, and family-level correlation matrices; there were no negative correlations identified above the species level profile (FIGS. 12A-12C). Regardless, all correlation matrices from the diversity population are primarily composed of positive pairwise correlation values, suggesting the microbial communities have co-evolved and stabilized in the sweetpotato plants, although some accessions deviate from this consensus. Negative correlations seem to track with occurrence of interactions between pathogenic that might have co-evolved with the plant but selected against by the plant and/or other microbial leaf endophytes. The exact-matching strain-level enhances taxonomic resolution by increasing the number of taxonomic observations, impacting the significance of putative microbial interactions, which allows researchers to better identify pivotal players within the microbial community.

### Example 10

Qmatey's shotgun sequencing benchmark with cross-reference filtering. Although evaluating Qmatey's performance with real sequencing data can provide biologically significant insights, it is difficult to accurately assess Qmatey's taxonomic classification without using a metagenomic dataset with known microbial composition. Using the Critical Assessment of Metagenomic Interpretation's (CAMI) shotgun data, Qmatey's taxonomic accuracy was compare using a set of classification performance metrics. CAMI recall is the total number of correct taxonomic matches relative to the total taxonomic matches within the gold standard or known community, and CAMI precision is the total number of correct taxonomic matches divided by all matches (including mismatches) within Qmatey's profile. Qmatey's recall at species-level was high at 0.9 compared to other tools that had a recall rate of between 0.0 and 0.4 (FIG. 9).

### Example 11

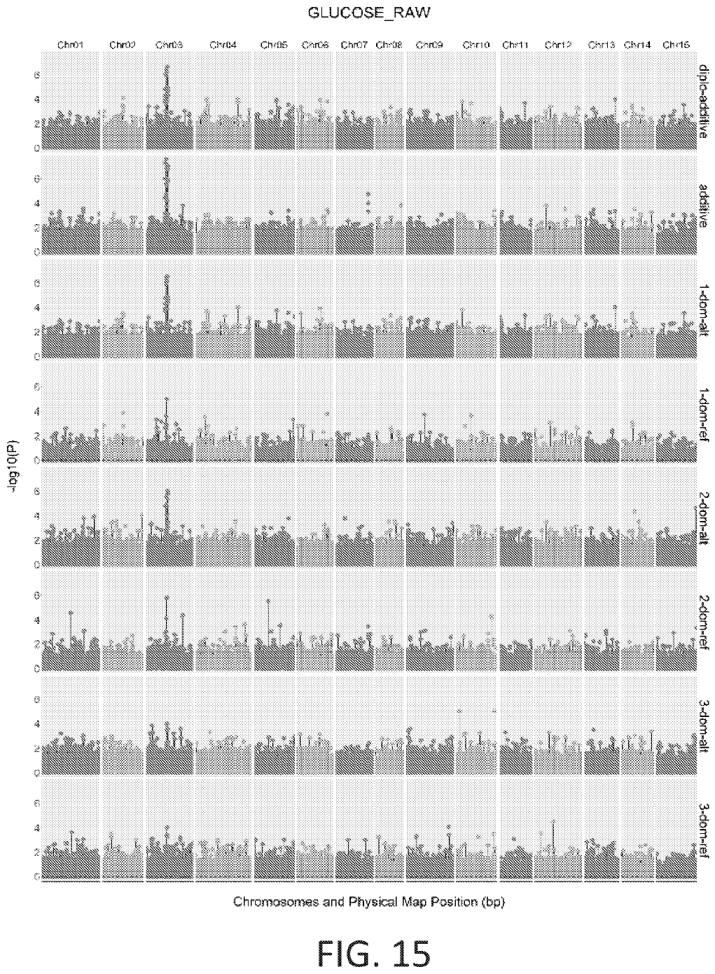

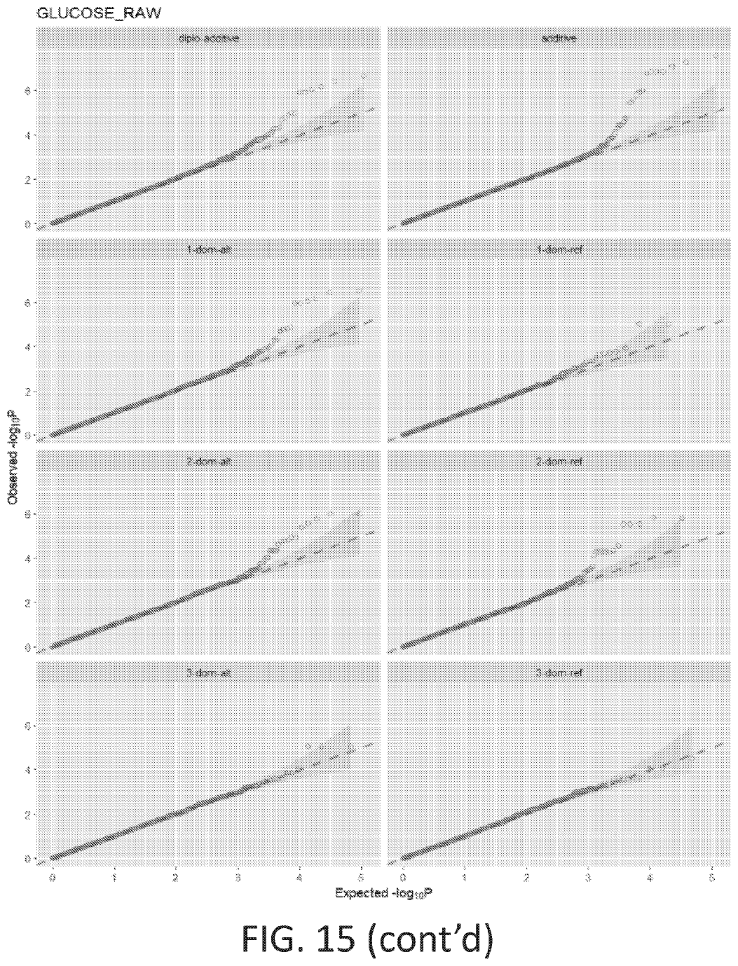

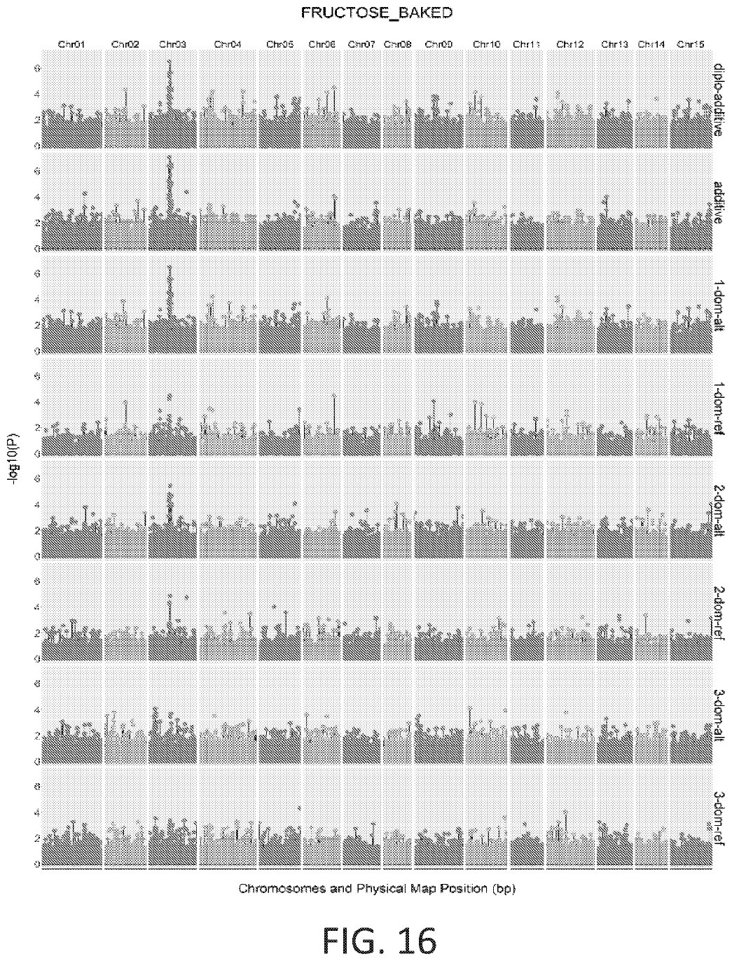

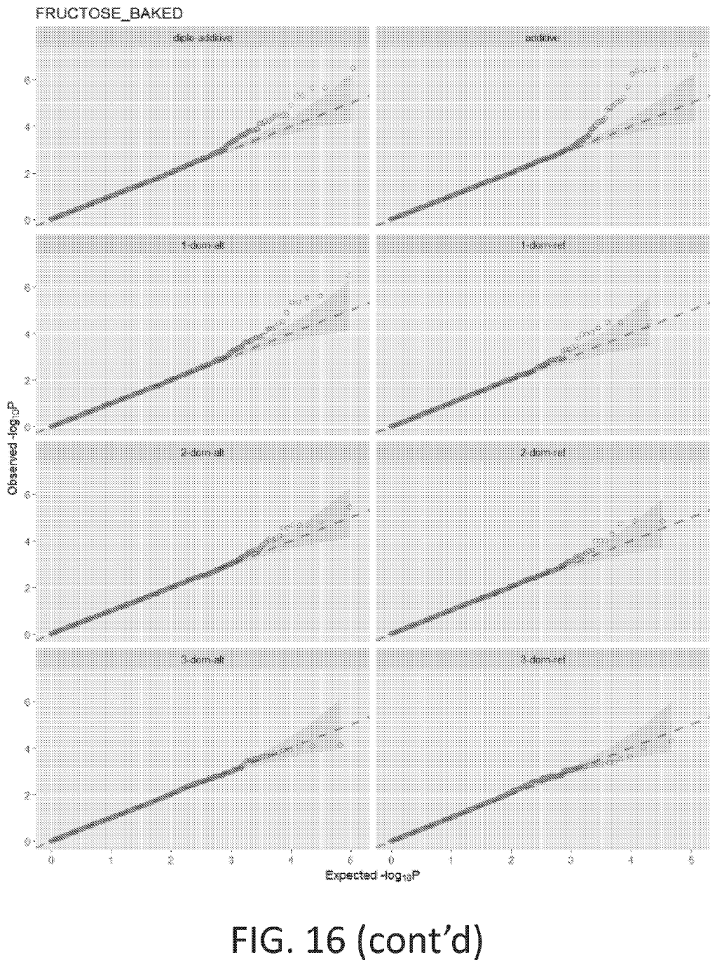

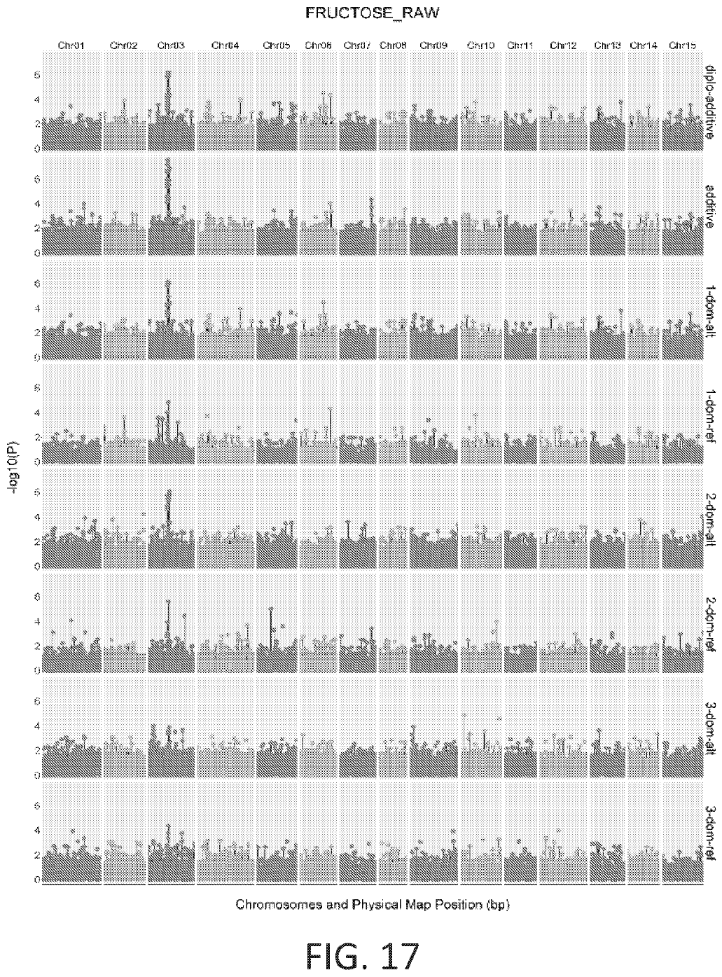

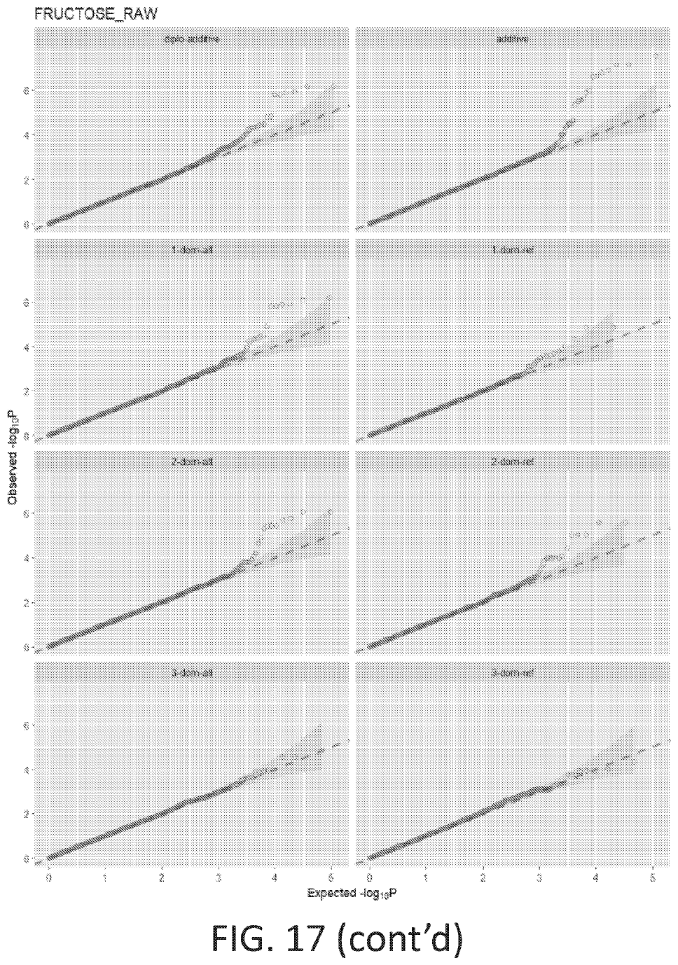

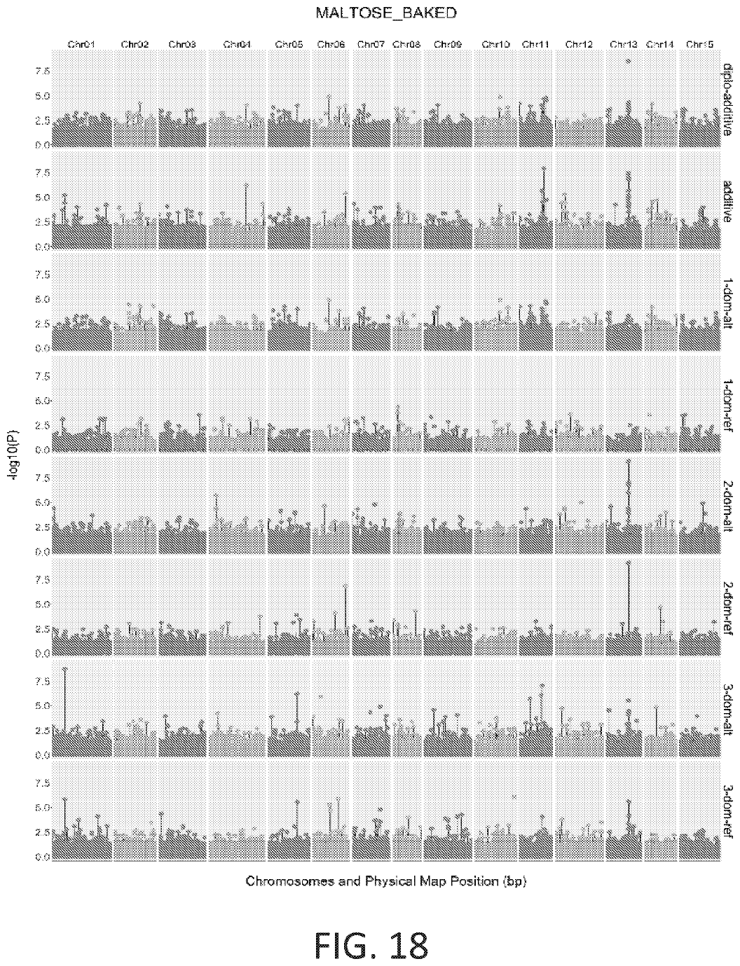

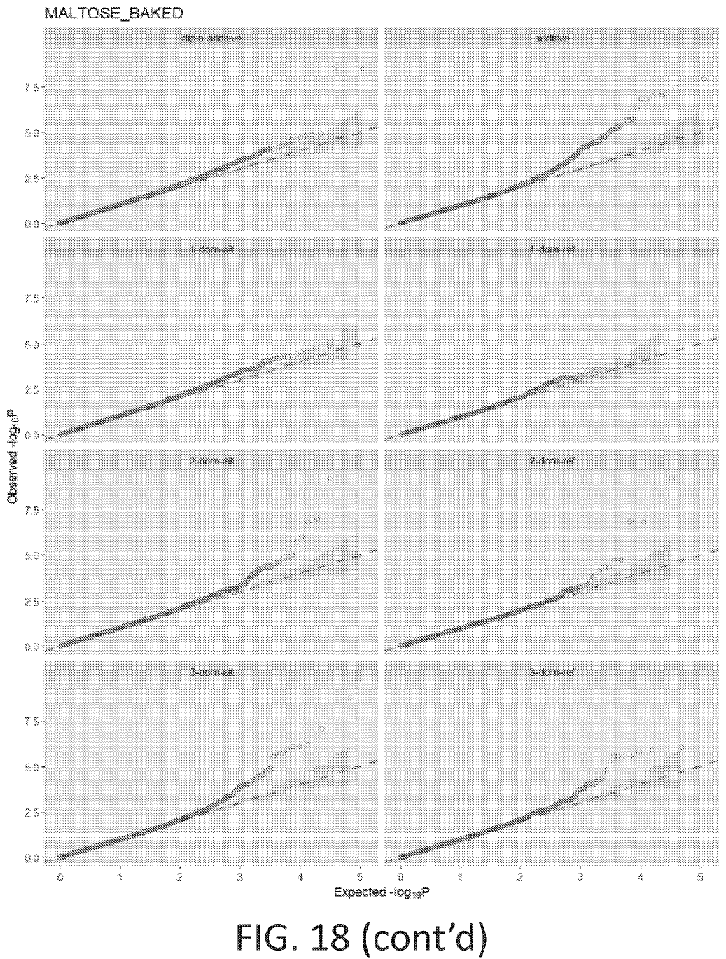

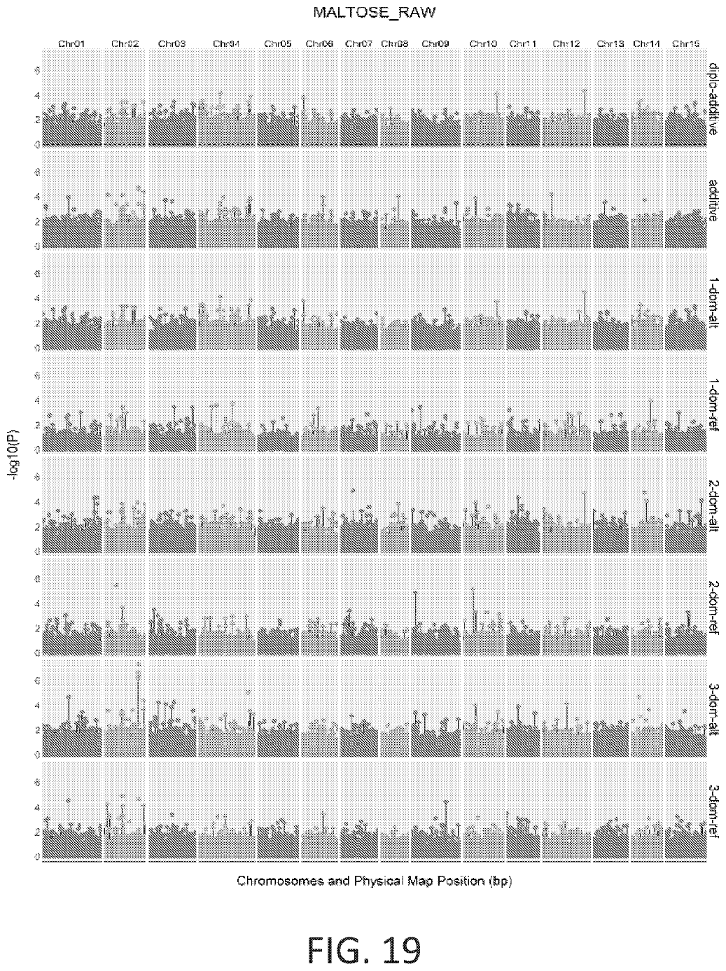

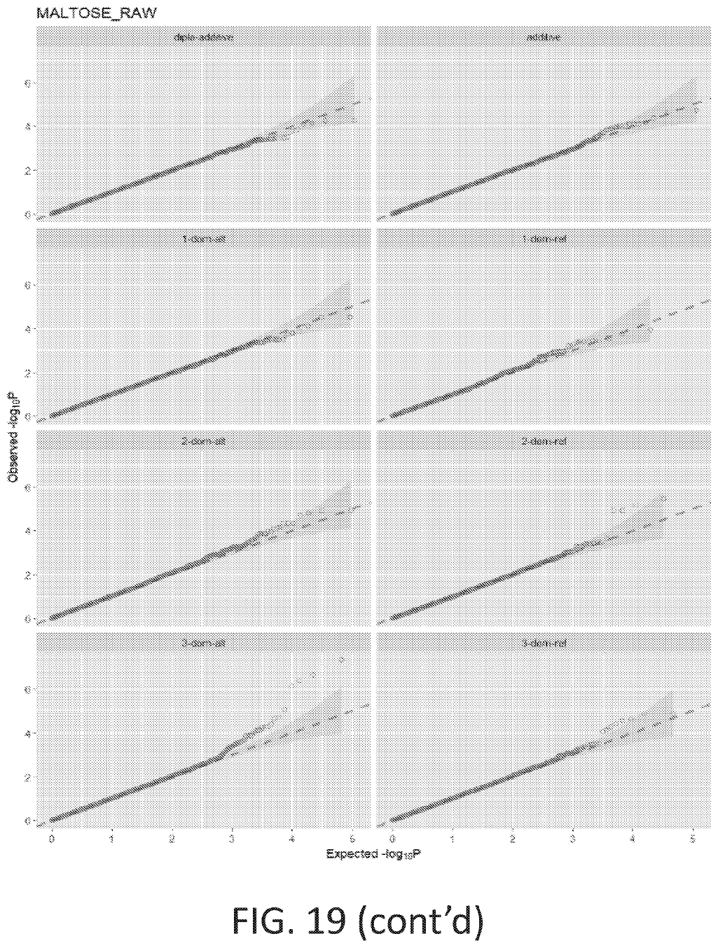

Traits with significant marker-trait associations were analyzed by generating a plot using Spearman's correlation for non-parametric data. The significant traits with the sugar profile include data on the concentration of raw maltose, reduced sugar total, total sugars baked, baked glucose, total hexoses baked, raw galactose, total sugars raw, and sweetness. The positive and negative correlation between traits can be seen in FIG. 13. Raw maltose has a slight negative correlation with raw galactose, total concentration of hexoses baked, and baked glucose. Reduced sugar total and total concentration of raw sugars has a slight negative correlation. All other traits have medium to strong positive correlations (see, e.g., FIGS. 14-19). These traits have significant associations with SNPs falling within or near candidate genes known to be involved in the following plant processes: abiotic and biotic stress response, intra- and intercellular transport, and cell sensing and signaling (FIG. 14).

The primary charts used to interpret the significant marker-trait associations are Manhattan plots and Q-Q plots. Dosage models (diplo-additive, additive, 1-dom-alt, 1-dom-ref, 2-dom-alt, 2-dom-ref, 3-dom-alt, 3-dom-ref) for this polyploid crop are represented in by individual Manhattan plots for each trait. The results indicate each trait has optimal allele amounts necessary to produce a crop with desirable sugar profile trait. The annotated genes and their putative functions are listed in Table 12 (below) and all Manhattan plots labelled with candidate genes can be made available upon request.

TABLE 12
Table of annotated candidate genes identified within the sugar profile. Twenty candidate
genes have been identified with putative function in the following cell processes:
abiotic and biotic stress response, plant growth and development, cell signaling,
inter- and intracellular transport, and DNA/RNA processing/expression.

| | | | | | | SNP |
|---|---|---|---|---|---|---|
| | | | Gene | | | proximity |
| | Putatuve | | dosage | −log₁₀P | | to gene |
| Ortholog | Function | Trait(s) | model | range | SNP ID | (bp) |
| Plant invertase | Hydrolyze | Fruc-R, | Additive | 6.49-7.59 | Chr03:11393679 | — |
| | sucrose into | Fruc-B, | | | | |
| | glucose and | Gluc-R, | | | | |
| | fructose. | Gluc-B, | | | | |
| | | Total | | | | |
| | | hexoses-B, | | | | |
| | | Total | | | | |
| | | hexoses-R | | | | |
| Nodulin MtN21/ | Amino acid, | Fruc-R, | Additive | 6.50-7.01 | Chr03:11867227 | — |
| EamA-like | auxin, and | Fruc-B, | | | | |
| transporter | sugar transport. | Gluc-R, | | | | |
| | | Gluc-B, | | | | |
| | | Total | | | | |
| | | hexoses-B, | | | | |
| | | Total | | | | |
| | | hexoses-r | | | | |
| 14-3-3 protein/ | Regulatory | Inositol-R | Diplo- | 6.81 | Chr12:2197520 | — |
| regulatory | protein that | | additive | | | |
| factor | binds diverse | | | | | |
| | signalling | | | | | |
| | proteins. | | | | | |
| Beta-Amylase | Hydrolyzes the | Maltose-R | 3-dom- | 7.31 | Chr02:15260226 | 5.532 |
| | α-1,4-glucan | | ref | | | |
| | linkages in | | | | | |
| | polysaccharides | | | | | |
| | to produce | | | | | |
| | maltose. | | | | | |
| Alkaline/Neutral | Hydrolyze | Maltose-B, | Additive | 6.19-7.45 | Chr13:18532390 | — |
| cytosolic | sucrose into | Total sugars- | | | | |
| invertase D | glucose and | B, sugar | | | | |
| | fructose. Part | equivalent | | | | |
| | of antioxidant | | | | | |
| | system for | | | | | |
| | cellular | | | | | |
| | reactive oxygen | | | | | |
| | species | | | | | |
| | homeostasis | | | | | |
| Hexose carrier | Sugar transport. | Fruc-R, | 2-dom- | 5.59-5.80 | Chr03_11589347 | — |
| protein/HEX6-like | | Gluc-R | ref | | | |
| Galactosyltransferase | Catalyzes the | Maltose-B, | 3-dom- | 8.72-8.75 | Chr01_5773824 | — |
| family protein | transfer of | total | alt | | | |
| | galactose. | reducing | | | | |
| | | sugars | | | | |
| Metallo-hydrolase/ | Involved in | Fruc-B, | 1-dom- | 6.15-6.49 | Chr03_11738891 | — |
| oxidoreductase | resistance | Gluc-R, | alt | | | |
| superfamily protein | against β- | Gluc-B | | | | |
| | lactams, which | | | | | |
| | are act | | | | | |
| | primarily as | | | | | |
| | inhibitors of | | | | | |
| | transpeptidases, | | | | | |
| | thereby | | | | | |
| | impairing the | | | | | |
| | synthesis of the | | | | | |
| | cell wall. | | | | | |
| Thioredoxin | Cytoprotective | Maltose-B, | Additive | 7.00-8.13 | Chr13_18684794 | — |
| superfamily | role against | total | | | | |
| protein | various | reducing | | | | |
| | oxidative | sugars | | | | |
| | stresses | | | | | |

### 5. MATERIALS AND METHODS

Calculating Empirical Base-Calling Error Rates in Barcode Sequences. All forward (R1) reads from the Miseq, HiSeq 2500 Rapid Run, HiSeq 2500 High Output, and NovaSeq datasets were subjected to multiple rounds of demultiplexing using the ngsComposer tool anemone.py. After first demultiplexing perfect matches, the unassigned reads were subsequently demultiplexed with a Hamming distance of 1. This process was repeated with unknown reads, increasing the mismatch value at each step until reads could no longer be identified having exceeded their inherent Levenshtein distance of 3. For each step, the reads in the resulting output files were compared base-by-base to their corresponding barcode sequence. On a per-position basis, the probability of error was calculated as the number of bases in that position that did not match the assigned barcode divided by the total number of reads assessed at that level of mismatch and fewer. The ASCII-encoded Q scores were counted and grouped in the same manner.

Comparing Performance of Motif and Threshold Filtering. For each of the datasets tested (Miseq, HiSeq 2×125, HiSeq 2×250, NovaSeq), reads were first trimmed of buffer sequences (6 bp) and demultiplexed with 1 mismatch using anemone.py, trimming successfully identified barcodes and pooling into a single R1 or R2 sample file. These pooled reads were used as the basis for downstream analysis of motif-detection and threshold filtering tools, as they are expected to begin with the RE digest motif immediately following the barcode sequence.

Filtering Methods. Filtering was performed using threshold-filtering (krill.py) and motif-filtering (rotifer.py). Under threshold filtering only reads consisting 90 percent of above of Q scores 30 or higher were considered passing. Motif filtering was performed in a library-specific manner. The Hiseq datasets were blunt-end fragmented using AluI/HaeIII digestion followed by A-tailing and adapter ligation. Only Hiseq reads beginning with the sequences “TCC” and “TCT” were considered passing. The Miseq and Novaseq datasets were prepared using the OmeSeq protocol (Olukolu 2020, unpublished manuscript) with NsiI/NlaIII digested reads. R1 reads in these libraries were searched for “TGCAT” and R2 reads were searched for “CATG”.

For each dataset, reads were first processed using threshold- and motif-filtering tools with failed reads written separately to file. For each of these output files, all passing and failing reads were processed again using the corresponding tool to be compared. For example, reads passing and failing the krill.py threshold filtering steps were then motif-filtered using rotifer.py, producing four final output files representing all combinations of pass and fail for combinations of the first and second tool.

Collapsing Unique Reads. After demultiplexing using variable length barcodes, reads within each library were end-trimmed to identical length before filtering to facilitate unbiased read comparisons downstream. After filtering methods were applied, all sequences were extracted from the fastq output using ‘sed’ commands. The ‘sort’ and ‘uniq’ commands were then applied to sequence files to collapse identical reads when possible. The ratio of collapsed reads to the total number of reads was calculated per-file.

Blastn Approach. Prior to motif or threshold filtering, reads were converted to fasta and queried against I. trifida/I. triloba/chloroplast reference assemblies using max_hsps=1 and max_target_segs=1 to return only the top-scoring alignment. Adapters were not removed prior to alignment to avoid inflating expect values (E-values) during threshold filtering, which is sensitive to read length. It was assumed that adapters would be evenly distributed among reads regardless of RE-digest presence or absence. The event of direct i5 and i7 adapter ligation with no insert is expected to be rare and in some library preparations in the datasets, impossible due to ligation incompatibility. E-values from the resulting .xml alignment were referenced to the original fastq file and were written to replace the read headers. If no alignments were found, the header was replaced with “na”. E-values are calculated using the reference database size, in this case 970,621,292 bytes.

Tool Overview. crinoid.py—Crinoid provides summary statistics on Q score and nucleotide distribution. Read and quality score lines of a fastq file are traversed k bases at a time. Each unique sequence of bases or ASCII scores are stored as a dictionary key. The total number of encounters with that sequence are stored in a list corresponding with the k walk position along the read. After all reads have been summarized, the positional information in the dictionary is converted to a matrix of 5×n for nucleotides and 41×n for Q scores, where n is maximum read length.

scallop.py—Scallop is a simple read trimmer and end-trimming tool. Fixed read positions at the front (-f) or back (-b) of the read are provided for manual trimming of reads. Users may also opt for quality-based end-trimming using a sliding window approach. In this setting, a window of fixed size (-w) walks base by base from 3′ to 5′ until the window contains only bases consisting of a given end-trimming Q score (-e) or higher.

anemone.py—Anemone demultiplexes single or paired-end reads using a tab-separated matrix of barcode and sample names. Reads are first examined for exact matches against the expected set of forward barcodes. To avoid possible I/O limitations of simultaneous file accession, the corresponding reverse reads are assigned in a separate pass. Although this creates some redundancy in processing, it allows for extreme flexibility in R1/R2 barcoding combinations (e.g. 96 forward and 96 reverse barcodes yielding 18432 paired output files are possible with anemone). Reads that are not assigned with exact matches (Hamming distance=0) are optionally subject to further passes through a lenient barcode search with greater leniency in Hamming distance, or mismatch (-m). In the instance that multiple barcodes match the queried read, the read is kept as an unknown to avoid sample misassignment.

rotifer.py—User-defined lists of sequences are used to search the start of forward and reverse reads for expected motifs. Motifs corresponding to the forward (-m1) and reverse (-m2) reads are expected in the beginning region of reads due to library construction using restriction enzymes and/or blunt-end A-tailing. Reads failing to contain these motifs are assumed to begin with sequencing error, have been incorrectly incorporated into the library, or were demultiplexed incorrectly. Paired end reads that both pass this test are kept in order and single ends that pass are output with a “se.” prefix.

porifera.py—A newline separated list of expected adapters (-a1) is provided by the user, optionally containing expected barcodes and restriction cut site motifs. Adapters are split into substrings of size k (-k) and each is stored in a dictionary of sequence and distance from the adapter start index. All k-mers of distance 0 are scanned for matches within the read, followed by k-mers of distance k, then 2k, and so on. This search process is repeated next with k-mers of distance 1, k+1, and repeats for rounds (-r) or until all k-mers are exhausted. K-mer matches pointing to the same start index are assessed per-read until a set of matching positions (-m) is reached or the adapter is assumed not to be present. An optional mode (-t) allows for a modified Smith-Waterman local alignment to be performed with t base overlap to qualify as a hit.

krill.py—Integer values for desired Q score (-q) and percent read composition (-p) are provided by the user for threshold filtering. ASCII characters at and above q are stored as a list of passing scores. For each read, a failing number of bases is determined by (100−p)*read length. Fastq Q scores are then tested 3′ to 5′ for membership in the pass list. If the non-passing characters exceed the failing number of bases, the read is rejected.

Pipeline Overview. The first step in the analytical pipeline entails normalization of the metagenomic data using a host, spike-in, or synthetic reference genome. This is only performed for libraries that have a spike-in standard and/or host genome in the case of endophytic microbiomes where the host genome represents a significant portion of the metagenome. The metagenomic data is aligned with the desired reference (e.g., host genome or spike-in standard) using BWA-MEM and processed with SAMtools and Picard.

For host genome-based normalization, an assumption is made that the microbiome constitutes a relatively uniform proportion of the tissue metagenome. Based on sequence alignment of leaf-derived sequence reads of 767 sweetpotato accession, less than 1% of the reads did not map to the plant reference genome. For normalization based on spike-in standards, a known and uniform concentration of the standard ensures normalization that accounts for bias during DNA extraction, DNA quantification, library preparation and sequencing. Regardless of normalization, all input files are compiled into FASTA format before aligning to a specified NCBI database via MegaBLAST (either locally or remotely) at a minimum e-value of 1e-10 with a maximum number of five subject sequences per query sequence. The nr database is used for both strain-level and OTU-based profiling. While the 16S/rRNA database can be used and is faster than the nr database, it was found that the nr database provides a better reference than the 16S/rRNA database (e.g., the 16S/rRNA is a subset of the nr database that does not capture of representative rRNA reference sequences).

After MegaBLAST, the metagenomic reads are compiled based on the taxonomic level of interest. If the user is interested in strain-level sensitivity, then an exact-matching algorithm is applied. For taxonomic levels at species level of higher, an OTU algorithm is implemented. For exact matching, metagenomic reads are stringently filtered based on query sequence alignment and compiling only reads that match the entire subject reference sequence with exact (100%) alignment. In addition to perfect alignment, query sequences are also filtered based on unique alignment (e.g., only reads that map uniquely to one taxonomic organism (diagnostic sequence) are subject to strain-level sensitivity). Taxonomic information is acquired for each read using the NCBI taxonomic ID and quantified based on the total number of reads associated with each organism. If the normalization factor was calculated previously, then the total amount of reads per taxa is multiplied by the sample-by-sample normalization factor. The average abundance value per taxa (strain) is calculated by dividing the total number of reads (e.g., sum of read depth across all diagnostic sequences mapping uniquely to each taxa) by the total number of the unique sequences. The quantification accuracy is calculated based on the standard error of abundance values (read depth) for unique sequences.

A taxonomic profile for all other taxonomic levels above strain (OTU-based) is based on a multi-alignment algorithm, which is different from the clustering algorithms used by other tools. Because taxonomic level identification is above strain-level, exact sequence match of the query sequence to the reference subject is not required, thus, a 97% sequence identify match is used for matching queries. If the read is aligned to a reference subjects within a single taxon, then the read is maintained, and the profile assigned to that taxon. For example, for genus level profiling, an input query sequence that aligns to several strains within the same genus, Fusarium, the read will be retained and assigned as a profile for Fusarium. On the contrary, if multiple strains map to two genera, Fusarium and Colletrichum, the read is discarded and not used for genus level profiling. After the multi-alignment algorithm is performed, all average abundance and standard error values for each taxon are computed in a similar manner as described for strain level.

Not only will the pipeline quantify and classify organisms within each sample, but it will also optionally perform a functional gene analysis for each dataset. To do this, all classified reads are aligned to the RNA reference sequence database via MegaBLAST. All reads that significantly aligned to coding regions with an evalue of 1e-10 are further annotated and directed to an optional output file from the pipeline. Furthermore, the cross-reference to the RNA reference database can be utilized as an additional filtering step. Reads that match to both databases that remain within the same genus are filtered into Qmatey's final output. While this might provide additional validation, it produces false negative rates since the nr database is more comprehensive than the RNA reference database.

Overall, the final output of the pipeline includes three datasets for each taxonomic profile: average number of reads, number of unique sequences, and standard read error per organism across all input samples. Optionally, a folder with annotated genes for every classified organism can be displayed. Each of these datasets is present at the desirable taxonomic level from strain to phylum. To visualize metagenomic data, a series of automated visualizations were developed.

Metagenomic Quantification. Once the reads are taxonomically classified, they are quantified using these metrics: average number of reads, unique sequences, and the standard error of the average read value. Each value is calculated for every classified organism across all input samples. These quantification metrics are impacted by the total read value, which is the total number of reads associated with the taxonomic organism. The total is multiplied by the optional, reference-based normalization factor calculated previously. Because genome size varies drastically across organisms, the total read value is not accurately representative of abundance. To account for genomic variation, the average read value is calculated by dividing the number of unique sequences (genes) by the total read value for each organism. These metrics are calculated for each taxonomic organism on a sample-by-sample basis, so the total read value of an organism in one sample is not impacted by the total read value in a separate sample, allowing Qmatey to analyze variance in microbial composition across all samples.

Leaf Microbiome Data Generation. The USDA's sweetpotato diversity population consists of 767 germplasm accessions that accurately reflect global crop diversity (Insert Library Prep Info: Bode). DNA was extracted from the leaves of each accession and sequenced with a quantitative reduced representation strategy. While the reads are predominantly derived from the sweetpotato genome, Qmatey's reference-based DNA normalization method allows for the extraction of endophytic, metagenomic data from each germplasm.

Shotgun Sequencing Benchmark. Benchmarking was performed utilizing the simulated, shotgun-sequenced low-complexity dataset from the Critical Assessment of Metagenomic Interpretation (CAMI, 5). Using the gold-standard taxonomic profile, binary classification values were calculated for each of Qmatey's taxonomic profile.

Software and Data Availability. The Qmatey pipeline is written in bash and R scripting languages (excluding dependencies). It is openly available on github (http://github.com/ryandkuster/ngsComposer) with a comprehensive set of example datasets differentiated by shotgun, amplicon, and reduced representation sequencing strategies. The whole genome sequencing data is a portion of the low complexity CAMI dataset available at (https://data.cami-challenge.org/participate), the amplicon sequencing dataset is a soil metagenome project derived from (link), and the reduced representation sequencing dataset is a portion of samples from the sweetpotato diversity population.

Phenotypic Data Generation and Exploration. The sweetpotato diversity panel is composed of 715 auto-allohexaploid (2n=6x=90) individuals from both the US sweetpotato germplasm collection maintained by the USDA, ARS, US Vegetable Laboratory (USVL) in Charleston, S.C., United States and the USDA, ARS, Plant Genetic Resources Conservation Unit (PGRCU) in Griffin, Ga., United States.

Researchers at the University of Georgia assessed the quality of storage traits in the USDA sweetpotato germplasm collection (Kays et al. 1998). Lines were grown at the University of Georgia Horticulture Farm in 1996 under standard commercial practices. At harvest, storage roots were cured 7 days at 29° C. and stored at 13° C. until further processed. For each trait measured, three storage roots per line were selected for sensory and chemical analysis.

The sugar profile is composed of 22 traits; however, only eight traits have at least one significant marker-trait association. To explore the relationship between those eight traits within the sugar profile, the following correlation analyses were performed. An eigenvector plot was generated to visualize the base for the AOE/PC clustering within the traits. To explore significant correlations between traits within the sugar profile, a correlation plot was generated where blue indicates positive correlations, red indicates negative correlations, and white indicates little to no correlation.

Genetic Material Generation and SNP Calling. Total genomic DNA was isolated from freeze dried leaf tissue using the DNeasy Plant Mini Kit (Qiagen). The integrity, purity, and concentration of the isolated genomic DNA was determined by 1% agarose gel electrophoresis and the florescence based PicoGreen dsDNA assay using a Synergy HTX Multi-Mode Microplate Reader. The genomic library preparation was performed using a modified genotype-by-sequencing (GBSpoly) protocol with the OmeSeq restricted representation sequencing method optimized for highly heterozygous and polyploid genomes as described by Wadl (2018). ngsComposer performed stringent quality filtering of raw sequence reads before SNP (Kuster et al., 2020).

The GBSapp pipeline was used for pre-processing raw fastq files, variant calling, and variant filtering (Wadl at el. 2018). The pipeline integrates various software, including GATK v3.7, optimized for highly heterozygous and polyploid species. The two physical reference genomes of sweetpotato's putative ancestral diploid progenitors, I. trifida and I. triloba were used for variant calling. Filtering parameters included read depth filtering for each data point and marker removal of markers. GBSapp generated 80,000 high quality, dosage-dependent SNPs with maf of 0.02 and no more than 20% missing data.

GWAS Performed using GWASpoly. GWASpoly incorporates 8 models of gene action and operates using optimized kinship models for hexaploidy sweetpotato (Rosyara et al. 2016). The results are interpreted using Manhattan plots and quantile-quantile plots (Q-Q plots) (Pearson and Manolio, 2008). Manhattan plots show significance level (−log-base-10 of p-value) for each SNP along the y-axis and the genomic position along the x-axis. The Bonferroni correction is used for significance to adjust for errors or false positives. Each dot represents a SNP in its location within the sweetpotato genome. The Bonferroni threshold runs horizontal on the plot with each SNP falling above it considered significant and thus necessary to further explore. The higher the SNP falls above the threshold, the stronger the trait-marker association. The Q-Q plots were used to explore the false positive rate of SNP associations due to confounding factors. The y-axis shows the significance level of association from least to greatest. The x-axis shows the SNP markers plotted against the expected distribution if there were no association. The SNPs that deviate from the expected distribution, are considered true associations. These plots provide a visual confirmation of significant SNP associations.

Identification of Candidate Genes. Using the significant trait-associated SNPs identified by GWASpoly, the physical genome assembly of the diploids I. trifida and I. triloba (http://sweetpotato.plantbiology.msu.edu/) were used as the reference genomes for identifying candidate genes. The putative function candidate genes were annotated using Michigan State University I. trifida JBrowse (http://sweetpotato.plantbiology.msu.edu/) feature. Additional gene annotations were performed using known gene function from the highly annotated genome of Arabidopsis thaliana as well as NCBI's BLAST (National Center for Biotechnology Information Basic Local Alignment Search Tool). Extensive literature review supports the proposed annotation of candidate genes.

### 6. SEQUENCES

The following nucleic acids are provided by the present disclosure, as referenced herein.

ssDNA adapter molecule
(SEQ ID NO: 1)
AATGATACGGCGACCACCGAGATCTACACTCTTTCCCTACACGACGCTC
TTCCGATCTn₄₋₆N₅₋₁₂VACGTA.
ssDNA adapter molecule:
(SEQ ID NO: 2)
GTGACTGGAGTTCAGACGTGTGCTCTTCCGATCT
n₄₋₆N₅₋₁₂CTAG.
Primer for probe and/or substrate binding:
(SEQ ID NO: 3)
CAAGCAGAAGACGGCATACGAGAAATGATACGGCGACCACCGAGATCTA
CACTCTTTCCCTACACGACG.
Primer for probe and/or substrate binding:
(SEQ ID NO: 4)
CAAGCAGAAGACGGCATACGAGATGTGACTGGAGTTCAGACGTGTGC.

It will be readily apparent to those skilled in the art that other suitable modifications

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the disclosure, may be made without departing from the spirit and scope thereof.

What is claimed is:

1. Forward and reverse single-stranded DNA (ssDNA) adapter molecules, the adapter molecules comprising:
(i) a probe binding region at the 5′ end of the adapters;
(ii) a buffer region distal to the probe binding region;
(iii) a barcode region distal to the buffer region; and
(iv) a restriction enzyme overhang motif at the 3′ end of the adapters.

2. The ssDNA adapter molecules of claim 1, wherein the restriction enzyme overhang motif comprises a nucleic acid sequence complementary to an overhang sequence produced upon cleavage by a restriction enzyme.

3. The ssDNA adapter molecules of claim 1 or claim 2, wherein the adapters are bound to a fragment of genomic DNA via complementation between the restriction enzyme motif of the ssDNA-adapters and the genomic DNA produced upon cleavage by the restriction enzyme.

4. The ssDNA adapter molecules of any of claims 1 to 3, wherein the restriction enzyme produces a 5′ overhang.

5. The ssDNA adapter molecules of claim 4, wherein the restriction enzyme is NsiI or NlaIII.

6. The ssDNA adapter molecules of any of claims 1 to 5, wherein the buffer region comprises a nucleic acid sequence from 4 to 8 base pairs in length.

7. The ssDNA adapter molecules of claim 6, wherein the buffer region comprises a nucleic acid sequence that is 6 base pairs in length.

8. The ssDNA adapter molecules of any of claims 1 to 7, wherein the barcode region comprises a nucleic acid sequence from 5 to 12 base pairs in length.

9. The ssDNA adapter molecule of any of claims 1 to 7, wherein the barcode region comprises a nucleic acid sequence from 7 to 10 base pairs in length.

10. The ssDNA adapter molecules of any of claims 1 to 9, wherein the buffer region is directly adjacent to the barcode region.

11. The ssDNA adapter molecules of any of claims 1 to 10, wherein the barcode region is directly adjacent to the restriction enzyme motif.

12. The ssDNA adapter molecules of any of claims 1 to 11, wherein the probe binding region facilitates binding to a substrate or probe.

13. The ssDNA adapter molecules of any of claims 1 to 11, wherein the probe binding region facilitates binding to a separate nucleic acid molecule that is complementary to at least a portion of the nucleic acid sequence of the probe binding region.

14. The ssDNA adapter molecules of any of claims 1 to 13, wherein the total length of the adaptor is from 25 to 100 base pairs.

15. A kit comprising any of the adaptor molecules of claims 1 to 14, for use in performing a sequencing reaction.

16. The kit of claim 15, wherein the kit further comprises at least one of:
(i) a buffer;
(ii) dNTPs;
(iii) a polymerase;
(iv) a restriction enzyme; and/or
(v) cos-probes.

17. A double-stranded genomic DNA fragment comprising the ssDNA adapter molecules of any of claims 1 to 14 appended to each end of the genomic DNA fragment.

18. A composition comprising a plurality of the genomic fragments of claim 17.

19. A solution-based array composition comprising a plurality of DNA complementary overhanging sequence probes (cos-probes) capable of integration into targeted regions of a genomic template, and the ssDNA adapters of any of claims 1 to 14.

20. The array composition of claim 19, wherein the cos-probes comprise at least one hairpin structure and an overhang complementary to the 5′ overhang of the restriction enzyme motif.

21. A quantitative reduced representation sequencing (qRRS) method comprising:
(i) appending the ssDNA adapter molecules of any of claims 1 to 14 to a plurality of nucleic acid fragments to form a nucleic acid library;
(ii) amplifying the plurality of nucleic acid fragments in the library using PCR and/or isothermal amplification;
(iii) hybridizing the library to a nucleic acid sequencing platform; and
(iv) sequencing the genomic fragments.

22. The method of claim 21, wherein the nucleic acids fragments have been digested with a restriction enzyme.

23. The method of claim 21, wherein the nucleic acid fragments are RNA or DNA molecules.

24. The method of claim 21, wherein appending the ssDNA adapter molecules comprises the use of cos-probes.

25. The method of claim 21, wherein the method results in at least 25% more sequencing reads.

26. The method of claim 21, wherein the method results in at least 50% more sequencing reads.

27. The method of any of claims 21 to 26, wherein the method comprises multiplexing.

28. The method of any of claims 21 to 27, wherein the method removes chimeric fragments caused by reconstitution of restriction enzyme sites.

29. The method of any of claims 21 to 28, wherein the method does not comprise PCR or ligation reactions.

30. The method of any of claims 21 to 29, wherein the method minimizes barcode swapping.

31. The method of any of claims 21 to 30, wherein the method enhances cluster generation.

32. The method of any of claims 21 to 31, wherein the method comprises quantification of allele dosage in diploid and polyploid organisms.

33. The method of any of claims 21 to 32, wherein the genomic DNA is obtained from one or more of bacteria, viruses, protozoa, plants, fungi, yeast, mammals, and any combination thereof.

34. The method of any of claims 21 to 33, the genomic DNA is obtained from a metagenome.

35. The method of any of claims 21 to 34, the genomic DNA is obtained from a microbiome.

36. The method of any of claims 21 to 35, wherein the genomic DNA is obtained from an organism having a polyploid genotype.

37. The method of any of claims 21 to 36, wherein the method comprises an error rate of less than 0.0002 across an entire length of a read.


SEQUENCE LISTING (4 sequences)

SEQ ID NO: 1 - DNA, 64 bp, Artificial sequence
  synthetic
aatgatacggcgaccaccgagatctacactctttccctacacgacgctcttccgatctvacgta

SEQ ID NO: 2 - DNA, 38 bp, Artificial sequence
  synthetic
gtgactggagttcagacgtgtgctcttccgatctctag

SEQ ID NO: 3 - DNA, 69 bp, Artificial sequence
  synthetic
caagcagaagacggcatacgagaaatgatacggcgaccaccgagatctacactctttccctacacgacg

SEQ ID NO: 4 - DNA, 47 bp, Artificial sequence
  synthetic
caagcagaagacggcatacgagatgtgactggagttcagacgtgtgc